(19) United States
(12) Patent Application Publication
(10) Pub. No.: US 2011/0208067 A1
(43) Pub. Date: Aug. 25, 2011

(54) Vascular patency management using electric fields

(75) Inventors: Carl Frederick EDMAN, San Diego, CA (US); Michael Wayne MACCOLLUM, Poway, CA (US); Darrel Dean DRINAN, San Diego, CA (US); Naresh Chandra BHAVARAJU, San Diego, CA (US)

(73) Assignee: PhiloMetron, Inc., San Diego, CA (US)

(21) Appl. No.: 13/030,925

(22) Filed: Feb. 18, 2011

Related U.S. Application Data
(60) Provisional application No. 61/338,457, filed on Feb. 19, 2010.

(51) Int. Cl. A61B 5/02

(52) U.S. Cl. 600/486

(57) ABSTRACT

The invention relates to the management of vascular patency by the use of implanted devices delivering one or more energies to a target vascular tissue wherein such energy delivery sources are substantially located in the vicinity of a target vascular region. The invention preferably employs electric currents as energy and utilizes one or more electrodes positioned in the vicinity of a target region and one or more electrodes located elsewhere. Such devices may be useful in the management of stenotic lesion formation adversely associated with a loss of patency in a variety of disease states or conditions, including vascular patency needed for hemodialysis used in the treatment of kidney failure.

### CROSS REFERENCE TO RELATED PATENTS

This application claims priority under U.S.C. Section 119(e) to provisional application No. 61/338,457, filed on Feb. 19, 2010.

### FEDERALLY SPONSORED RESEARCH OR DEVELOPMENT

Not Applicable

### BACKGROUND OF THE INVENTION

1. Field of the Invention

The present invention relates to the management of vascular patency by the utilization of one or more energies delivered by an implantable device. More particularly, the present invention relates to use of an implantable device preferably utilizing electric currents to manage vascular patency. Such devices may also include one or more sensors intended for the monitoring of patency to better enable the therapeutic application of one or more energies in the management of patency.

2. Background

Vascular structures may experience diminished patency as a result of naturally occurring processes or from the body's response to introduced materials or devices. In many instances, diminished patency may be attributable to formation of a stenosis lesion, which in turn disrupts flow, e.g. patency, through the luminal space.

A common example of such diminished patency is that arising from a stenosis associated with the presence of a vascular stent. That is, when a blood-carrying vessel, e.g., an artery or vein, experiences a flow constriction, it is a common medical practice to insert an expandable tube into the blood vessel to counteract the constriction and re-establish the blood flow. Such an expandable tube, commonly called a stent, is often in the form of plastic or metal wire mesh. Initially, the mesh tube is collapsed into a small diameter and is then expanded by a variety of methods after insertion into the blood vessel. After expansion, the stents are affixed to the vessel wall through radial tension. Stents are used extensively in cardiovascular applications, especially for treating coronary artery blockages.

However, vascular stents and associated angioplasty procedures may result in a thickening of the inside the vessel wall. At times, the tissue growth can be so severe that it can result in restenosis—a re-closure of the vascular expansion created by the angioplasty and supported by the stent. While some improvements, such as drug-eluting stents, have shown to slow the tissue growth, the efficacies of these remedies are limited. Therefore, a need exists for a new approach for retarding restenosis that would substantially extend the usable life of implanted vascular devices including a stent.

Likewise, stenosis and accompanying loss of patency is frequently observed following the introduction of a graft or shunt between two blood vessels such as an arteriovenous graft (AV graft). These grafts are routinely employed for vascular access during hemodialysis. However, PTFE grafts exhibit poor primary patency rates, e.g. approximately 50% at year 1. Primary patency rates of PTFE dialysis grafts are believed to be significantly reduced by thrombotic events related to stenosis resulting from neointimal hyperplasia at the venous anastomosis. Therefore, improvement in the patency by reducing stenosis is needed to extend the average useful lifetime of PTFE grafts.

Loss of patency occurs with other vascular processes and procedures and therefore, the ability to manage patency in a variety of vascular applications represents a significant medical need. Unfortunately, no one method or approach appears to adequately address the challenges of vascular patency management.

For example, vascular grafts having added endothelial cells have been suggested as an alternative approach to maintain patency, e.g. U.S. Pat. No. 5,723,324 to Bowlin, et al; U.S. Pat. No. 5,674,722 to Mulligan, et al., U.S. Pat. No. 5,785,965 to Pratt, et al., and U.S. Pat. No. 5,766,584 to Edelman, et al. Although these approaches may have the potential of providing improved patency over naive grafts in certain instances, these approaches still faces many hurdles to enable successful implementation. First, it is desirable that the cells used to seed the graft be autologous or otherwise non-immunogenic to avoid recognition and destruction by the patient's immune system. To obtain autologous endothelial cells from a patient, the cells must be harvested from an isolated blood vessel. The harvesting surgical procedure not only increases prosthetic implant preparation time, but can also lead to complications and discomfort for the patient.

Accordingly, the need remains to identify an approach that enables mitigation of the vasculature's response to vascular procedures and/or implanted devices and thereby maintains patency of the vasculature at or near the site of such activities. Towards this end, continuous electric fields have been noted to affect the migration of certain vascular cell types in vitro, e.g. Bai, et al. (2004) Arterioscler Thromb Vasc Biol vol 24, pp 1234-1239. Using a different approach, Burwell et al. (U.S. Pat. No. 7,730,894) teach that photonic irradiation may be employed to advantageously affect vascular tissue in photodynamic therapy, however, the method taught is not applicable for extended use in vivo and requires additional agents. Therefore, the need remains for an approach that enables the effective management of vascular patency.

### SUMMARY OF THE INVENTION

The present invention claims the novel method of managing patency in a desired fashion through the controlled application of one or more energies delivered by an implanted device in substantial contact with vascular tissue or region by the application of these energies over an extended period of time. In a preferred embodiment of the invention, such energies are in the form of controlled application of electrical currents which produce electrical fields of defined strength and orientation in a targeted vascular tissue or regions.

In order to accomplish the preferred embodiment, devices of the present invention include at least one first electrode having a first surface at or near a vascular region to be managed; at least one second electrode located elsewhere; and are so configured to enable delivery an electric current which is intended to pass through body tissue or biological matter interposed between said first electrode and said second electrode for the purpose of controlling vascular tissue response in the vicinity of the first electrode.

Accordingly, devices of the present invention utilizing such electrodes also have necessary circuitry and power to enable the delivery of such electric currents in a controlled fashion. In preferred embodiments, such devices utilize structures having electrodes first surfaces positioned in substantial contact with the exterior surface of a blood vessel, e.g. by having electrodes located in a structure that is in the form of sheath or wrap positioned about the vessel, and thereby enabling the passage of an electrical current from at least one first electrode through a vessel wall into the blood stream and then again through a vessel wall to at least one second electrode.

In yet other embodiments of the present invention, at least one first electrode and at least one second electrode are contained within a synthetic vascular structure, e.g. a synthetic graft, such that the first surface of the first electrode is in substantial contact with the blood passing through the synthetic structure.

In still other embodiments of the present invention, electrodes of the invention are located within structures positioned within the lumen of a vessel such that in operation at least one first electrode is in substantial contact with the interior portions of the vascular lumen.

In alternate embodiments of the present invention, energies utilized for patency management may be of forms other than or in addition to those of electrical currents. Such other forms may include, but are not limited to photonic, electromagnetic, acoustic, mechanical, chemical or thermal energies. In such embodiments, one or devices enabling the delivery of such energies in controlled fashions may be utilized at or in the vicinity of the vascular region to be managed having one or more elements, e.g. light sources, so configured as to enable the controlled delivery of the energy to the vessel or tissue region.

In yet other embodiments of the invention, one or more sensors may be employed to monitor the vascular patency status and thereby enable guidance for the controlled application of one or more energies to manage vascular patency. Such sensors may utilize one or more energies that are employed to manage vascular patency as part of sensing activities. In certain forms of embodiments of the present invention utilizing electrical currents to manage patency, one or more electrodes employed for the delivery of electric currents for managing patency may also be employed in the sensing of vascular patency through impedance measurements, i.e. the location, occurrence and/or magnitude of vascular patency change.

### BRIEF DESCRIPTION OF THE DRAWINGS

FIG. 1—Representational illustration of a preferred embodiment of the present invention.

FIG. 2—Cross-sectional illustration of elements of a stent-like alternative embodiment of the present invention.

FIG. 3—Side-view illustration of vascular structure and electrodes and structures of the present invention.

FIG. 4—Illustration showing electrodes and structures of the present invention axially arranged on a vessel.

FIG. 5—Illustration showing a spiral structure for the positioning of electrodes of the present invention.

FIG. 6—Side view of blood vessel and electrodes of the present invention located on outer aspect of vessel. Panel A also showing electrical path in absence of high resistivity structures about electrodes; Panel B showing electrical path in presence of high resistivity structures about electrodes.

FIG. 7—Illustration of an embodiment of the present invention configured to enable use of conductive polymers or gels with insert showing expanded view of box-like construction for holding conductive gels.

FIG. 8—Side-view illustrating an embodiment of the invention employing conductive and nonconductive hydrogels as elements of electrodes and structure of the present invention in contact with vessel.

FIG. 9—Figure illustrating an example of an embodiment of the present invention utilizing serpentine electrode wires contained in supporting mesh.

FIG. 10—Side-view of a vascular graft structures incorporating elements of the present invention. Panel A showing electrodes positioned within a synthetic vascular structure. Panel B showing electrodes positioned within a synthetic vascular structure and electrodes positioned about a vessel.

FIG. 11—Cross-sectional view of two concentric stent-like electrodes radially-separated by an insulating layer according to one embodiment of the present invention.

FIG. 12—Cross-sectional view of two concentric and radially separated stent electrodes made from dissimilar metals according to an embodiment of the present invention.

FIG. 13—Cross-sectional view of an implantable vascular device having axially-separated stent electrodes according to an embodiment of the present invention.

FIG. 14—Cross-sectional view of an implantable vascular device having a stent-electrode and a non-stent electrode. Panel A showing non-stent electrode placed with vascular lumen according to an embodiment of the present invention. Panel B showing non-stent electrode placed outside of vascular structure.

FIG. 15—Cross-sectional view of an embodiment of the present invention of a stent-like structure having capacitor elements for power.

FIG. 16—Diagram of one embodiment of control circuitry for the generation of pulsatile electrical currents for use by the present invention.

FIG. 17—Illustration of one embodiment of the present invention showing the implanted device, external communication source and remote data base.

### DETAILED DESCRIPTION OF THE INVENTION

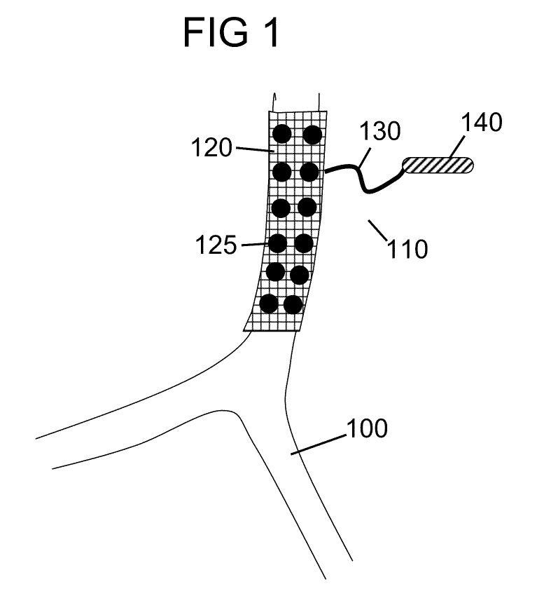

The invention generally relates to the novel application of one or more energies for the management of vascular patency. A general illustration of a preferred embodiment of the present invention is presented in FIG. 1. As shown, device 110 has structure 120 in which electric current delivery sources 125, e.g. electrodes, are positioned. Within the scope of this embodiment, electrodes 125 may serve as first or second electrodes, according to the mode of electrical activation applied. In preferred embodiments, a first surface (not shown) of electrodes 125 is in substantial electrical contact with the outer aspect of vascular structure 100. Supporting structure 120 is configured to be substantially tubular when positioned about the vessel, i.e. when wrapped or placed about vascular structure 100. Prior to positioning about a vascular structure, structure 120 may be flexible and may constructed in a variety of shapes and dimensions, e.g. sheet-like contiguous structures or mesh like structures with one or more openings or gaps in the surface, in order to accomplish the method of the present invention. Upon positioning about the vascular structure, e.g. a blood vessel, structure 120 may be held in place using sutures, clips or ties that circumferentially pass around the structure and vessel contained therein.

Within the broader context of the present invention, structure 120 preferentially has a plurality of energy delivery sources, e.g. electrodes, such that energies released from one or more energy sources effectively transits into at least a portion of the vascular tissue structure to effect desired patency management.

Additional elements to enable the controlled delivery of energies such as electrical currents, includes control circuitry and power source (battery) as well as possible communication means, e.g. radio transceiver and antenna. Such elements are preferably contained within circuit module 140. Circuit module 140 is electrically connected to electrodes 125 by insulated wires 130 or other forms of electrical connection, e.g. printed circuitry, to enable delivery of electrical currents to one or more first or second electrodes. In preferred embodiments, such circuit modules are implanted within the body however within the scope of the invention, external placement of the controlling circuitry module, e.g. employing percutaneous wire connections through the skin are conceivable.

Circuit module 140 is configured to protect electronic components contained within from exposure to the body environment (and vice versa). Module 140 may be constructed in a variety of fashions and materials, e.g. as a rigid box made with biocompatible plastics or metals, e.g. medical grade stainless steel or titanium. In alternate embodiments, circuit module 140 may be a cast or formed structure about circuitry, and constructed using pliable biocompatible casting materials, e.g. silicone. In yet other embodiments, the circuit module and elements contained within may be incorporated or directly affixed to structures of the present invention utilized to delivery energies to vascular tissue 100, e.g. as modules incorporated in structure 120 or structures such as synthetic grafts or other vascular devices.

Components comprising circuit and power elements may consist of one or more variety of electronic components, e.g. resistors, transistors, amplifiers, integrated circuitry, and/or components substantially constructed utilizing printed electronic circuitry fabrication technics, according to the embodiment of the invention. Likewise, devices of the present invention may be powered in a variety of means, e.g. lithium batteries, remotely rechargeable batteries having one or more radiofrequency energy absorbing antennas, etc.

In related embodiments, a plurality of circuit modules may be employed such that selected elements are located within one or more modules, e.g. a power module separately disposed from a circuitry module. Such embodiments may facilitate replacement of one or more circuitry elements upon need, e.g. to facilitate replacement of implanted power modules without substantive disruption of other connections, wires, etc. connecting to electrodes.

All materials comprising the device of the invention that are in contact with surrounding body tissues and fluids are preferably composed of biocompatible materials and also preferably configured to prevent unwanted penetration of body fluids into interior aspects of the device, e.g. into circuitry components and/or connections. In addition, devices of the present invention are preferably composed such that these may be sterilized prior to implantation into a mammalian body and thereby minimize infection risk.

In other embodiments, the device of the present invention or elements of the present invention such as electrodes may be substantially incorporated into structures such as grafts, stents, or supports that afford additional medical purposes beyond those described in the present invention. In still other embodiments, the device of the present invention or elements of the invention may be positioned within a vascular structure, e.g. within the lumen of a vessel. In such embodiments, the device and methods of the present invention may be incorporated in whole or in part into other forms of medical devices having additional functionalities and/or uses, e.g. stents or grafts.

Construction of the electrodes, structures, circuit modules, circuitry and powering systems according to these various embodiments are well known to those skilled in the art of medical electronics and implanted devices and the scope of the present invention is not restricted to any one form or type of structure, electrode, module, circuitry and/or power.

In the context of the present invention, vascular tissue includes, but is not limited to, blood vessels, biological structures for conveying biofluids such as urine or lymph, e.g. ureters, vascular implants, and native tissue and/or synthetic grafts. Vascular patency in the context of the present invention refers to the flow of blood or other body fluids through a vascular tissue in a manner consistent with desired functionality. Desired patency functionality may include the preservation of existing blood or fluid flows at a levels sufficient to maintain desired bodily functions, e.g. the delivery of oxygen and nutrients to tissue regions and/or the removal of waste compounds or materials from tissue regions. In other uses, functionality may include blood or fluid flows enabling one or therapeutic procedures to be accomplished, e.g. blood flow within a vascular access point sufficient to enable hemodialysis treatment. In yet other forms, patency functionality may also include desired remodeling or change in vascular structure, accelerated healing or improved cellular composition of the vasculature in the vascular tissue region. The scope of the present invention is not limited to any one form or type of vascular patency and/or functionality.

In the context of the present invention, management of vascular patency may include, but is not limited to, the guidance of the motility of and/or regulating the proliferation of one or more cells types involved in normal vascular function, e.g. endothelial cells lining vascular luminal spaces, or intimal cells responsible for vessel wall integrity and resiliency, and those cell types whose infiltration or proliferation in vessel walls and/or luminal regions results in non-desired obstruction of flow in the luminal region. Such cell types may include cell types such as fibroblasts, smooth muscle cells or inflammatory cells. Management may include direct actions upon one or more of the above cell types, or indirect actions, e.g. actions mediated by one or more extracellular factors or other cell types upon one or more of the intended target cells and the scope of the invention is not limited to any one form of cell type or mode of interaction with biological tissues.

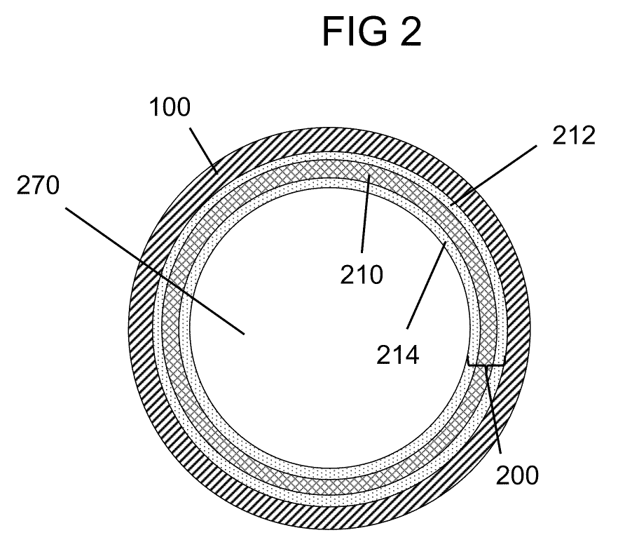

By way of example of an alternate form of the present invention, FIG. 2, illustrates a medical device, e.g. a stent 200, located in a vessel lumen having electrodes and other structures of the present invention incorporated. As shown, device 200 is comprised of an expandable metal mesh tube 210 surrounded by an outer semipermeable layer 212 and an inner semipermeable layer 214. In this embodiment, at least a portion of metal mesh tube 210 also serves as a first electrode of the device. Upon activation, an electric current passes from a first surface of the first electrode of the metal mesh tube to a second electrode (not shown) thereby completing the electrical circuit. In the process, the electric current may traverse through the outer semipermeable layer 212 and through the wall of vascular structure 100, or alternatively, may traverse through the inner semipermeable layer 214 and the blood stream 270. In both cases, semipermeable layers 212, 214 may serve to distance the first electrode from adverse direct contact with the surrounding vascular structure 100 and/or blood stream 270.

The use of one or more applied energies for the purpose of managing vascular patency is substantially different from passive technologies utilizing materials and surface textures that are intended to minimize body's adverse reaction to implanted materials, e.g. surface modification. Accordingly, devices of the present invention may employ one or more of these other passive technologies in order to better manage vascular patency and/or minimize body reaction by the body to one or more components of the present invention.

In various embodiments of the present invention, one or more sensing technologies substantially located in the target vascular region may be utilized to monitor vascular patency in said region wherein such determination may be utilized in the control of the delivery of one or more therapeutic energies. Such sensing technologies may include sensing technologies employing electrical, electromagnetic, mechanical, photonic or acoustic signals and the scope of the present invention is not limited to any one form of sensor or sensor energy.

Aspects of the present invention may be related to the methods and devices described in US patent: “GATEWAY PLATFORM FOR BIOLOGICAL MONITORING AND DELIVERY OF THERAPEUTIC COMPOUNDS” (U.S. Pat. No. 7,044,911); and US patent applications: “USE OF ELECTRIC FIELDS TO MINIMIZE REJECTION OF IMPLANTED DEVICES AND MATERIALS” (Ser. No. 10/722,306) and “FOREIGN BODY RESPONSE DETECTION IN AN IMPLANTED DEVICE” (Ser. No. 11/862,069), which are incorporated by this mention in their entirety herein.

A more detailed description of selected aspects of the present invention is presented below.

Energies In general terms, the present invention utilizes the controlled delivery of one or more energies in the management of vascular patency. Such energies may include electrical, electromagnetic, photonic, acoustic, chemical or physical energies that are delivered at levels or intensities not intended to result in the immediate destruction or disruption of vascular cells and/or surrounding tissues. This novel approach contrasts to other systems utilized to manage vascular patency, such as thermal ablative technologies, that may raise cellular temperatures, disrupt cellular structures, e.g. membranes, or otherwise result in immediate death or disruptive conditions to targeted cells and tissues.

In addition to the levels of energy delivered, the delivery of energies within the scope of the present invention preferentially occurs over extended periods of time, e.g. hours, days or weeks, to accomplish the intended management of patency. This delivery period contrasts relatively short application periods, e.g. seconds or minutes, of those forms of patency management technologies relying on the immediate destruction or disruption of one or more cell types and/or tissue structures. In such disruptive technologies, continued application of the disruptive energies for extended time periods may result unwanted disruption or destruction of tissue beyond the targeted region.

In preferred embodiments, the invention described herein uses applied electric currents for the management of vascular patency wherein the controlled application of an electric current is intended to result in a desired therapeutic outcome. For example, the applied electrical currents may result in a directional electrophoretic movement of one or more charged species and/or the guidance of one or more motile cell types responsive to these electric fields, i.e. galvanotaxis, or the alteration of cellular proliferative activity in a region in the vicinity of a first electrode thereby resulting in a desired therapeutic effect.

Accordingly, elements associated with a preferred embodiment of the present invention are one or more first electrodes located in or about the vascular region where the management of patency is desired, one or more second, i.e. counter, electrodes located elsewhere, e.g. in another region either in vascular or in body tissue, and the electrical activation of at least one first electrode and at least one second electrode thereby resulting in a directional passage of an electrical current between said first and second electrodes.

Advantageous use of electric fields to control patency according to the present invention may be accomplished either before an unwanted patency event occurs or in response to an unwanted patency status. For example, one such use might be the prophylactic application of electric fields. Such prophylactic application may serve to minimize the initial migration of cells prior to an undesired outcome, e.g. a stenotic growth arising at a site of vascular surgery. An alternative use of electric fields would be to advantageously accelerate the movement of desired cell types to vascular regions that may result in improved vascular performance. An example of such desired movement of cells into a region may be the enhancement movement of vascular endothelial cells into injured regions such as those injuries arising from angioplasty and/or on to artificial surfaces such as grafts or stents and thereby provide an environment more conducive to unimpaired vascular functionality.

To control the migration or behavior of select cell types in the vicinity of critical vascular regions and thereby manage patency, one or more first electrodes in close proximity to critical region may serve as an anode (positive bias) with one or more second electrodes (counter electrodes) serving as the cathode (negative bias). This may be useful if the directionality of the galvanotactic movement of a targeted cell type is according to the polarity of the electric field thus established. In alternate embodiments of the invention, the polarity of the electrodes may be reversed to address other cell types and/or cellular activities. In still other embodiments, the polarity may be alternated between one or more sets of electrodes. In yet further embodiments, a multitude of electrodes may be successively activated including the use of sets of electrodes with alternating polarity such that specific electrodes (or electrode regions) may alternatively function as either cathode or anode depending upon the desired therapy.

In yet other embodiments of the present invention, the relative areas of the first surface of the first and second electrodes may differ. Such differing areas may be achieved directly through the dimensionality of electrode sizes as constructed or actively altered through use of simultaneous activation of two or more electrodes. In general terms, differing electrode areas enables additional control of the density of the electric field in the immediate vicinity of one or more electrodes thereby enabling further control of tissue responses in these areas.

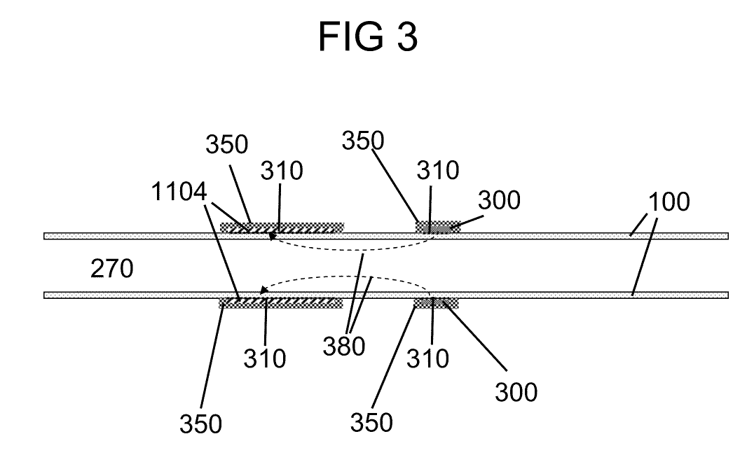

A simple illustration of one form of an embodiment where first surfaces of electrodes have different areas is shown in FIG. 3. In this embodiment both the first electrodes 300 and second electrodes 320 are positioned on the outside aspect of the blood vessel 100. An insulating structure, 350, serves to limit current flow through surrounding tissue 380, thereby directing the current flow preferentially from first surface 310 on first electrode 300 through vessel wall 100 through luminal space 270 then through vessel wall 100 to first surface 350 on second electrode 320, as indicated by dashed arrow 380.

In such applications, advantageous use may be made of first electrodes having dimensions differing significantly from second electrodes. In particular, the second electrodes may be significantly greater in area than the first such that the electric fields in the vicinity of the second electrodes do not result in a biologically active response, e.g. the guiding of cellular motility, thereby allowing only the first electrodes to exert vascular tissue control. In a further refinement of the invention, the second electrode may be positioned in a non-vascular region thereby further lessen the impact of electric field on vascular tissues in the relative vicinity of the second electrode.

Alternatively, a device of the present invention may employ electrodes of similar sizes, each of whose activation is adjustable or otherwise switchable. Activation of one electrode as a first electrode and concurrent activation of two other electrodes as second or counter electrodes will therefore result in the electric current density in the immediate vicinity of the second electrodes being approximately half the density in the vicinity of the first electrode. This difference in electric field density is attributable to the relative areas of the electrodes.

This attribute of adjustable electrode area enables embodiments of the present invention wherein a desired polarity of current at an intended current density having a desired electric field strength is delivered to tissue in the vicinity of select first electrodes, whereas a different, e.g. lesser, ineffective, current density having an electric field of opposing orientation is present at the corresponding second or counter electrodes. In combination with switchable polarity of current application enables effective coverage, e.g. first electrodes, over regions of devices having a plurality of electrodes by adjustment of electrode areas and polarities.

In general embodiments of the invention employing electrical currents and electrical fields to enable patency management, electric field strengths, resultant from the passage of a delivered electrical current in the target vascular tissue regions and/or adjacent body fluids, are preferably between 0.1 V/cm and 20 V/cm, more preferably between 1 V/cm and 5 V/cm where such field strengths are governed in part by the electric current densities in these regions. Other electric field strengths may be utilized in selected embodiments of the invention. Within the scope of the invention, the applied currents generate such local field strengths preferably within 10 mm of the first electrode first surface, more preferably within 1 to 2 mm of the first surface. Such considerations are dependent on the geometry and dimensions of the electrode and of the vascular tissue or structure as well as of the intended use or application, e.g. mitigation of lesion formation in a fistula versus a graft.

Overall, the exact form, amplitude and polarity of the currents applied and the nature of any additional technologies employed are determined by the application, the tissue/cell types involved and the functional requirements of the medical device. As the reader may well appreciate, a variety of electrode dimensions, arrangements, and activation configurations are conceivable within the scope of the present invention and the scope of the present invention is not constrained by the examples presented herein.

Within the scope of the present invention, materials that comprise substantial portions of first and second electrodes preferentially are electrically conductive and biocompatible. Such materials may include, but are not limited to, noble metals and metal alloys such as platinum or platinum-iridium amalgams, conductive polymers, gels or epoxies, and/or conductive plastics. In general, the form of the electrode will preferentially enable contact of a first surface with the intended vascular region or tissue. A variety of electrode forms are potentially employable, dependent on the embodiment of the invention, including planar, wire, mesh or other structures enabling electrical current introduction into tissue.

In various embodiments where electrodes are substantially planar in configuration prior to placement about a vessel, such electrodes may be constructed, e.g. be thin enough, to be pliable to vascular movement and/or conforming to the movement of the underlying vessel to activities such as pulsation. For example, semi-circumferential planar platinum-iridium alloys, 90/10 in composition, that are 1 mm wide by 3 mm long and having a thickness of approximately 100 um may have suitable flexibility to accommodate 10% diameter changes with minimal constriction on an underlying blood vessel of approximate 2 mm in diameter.

Conductive polymers utilized as electrodes may be advantageous in certain embodiments. In particular such electrodes may provide a relatively larger first surface area as compared to a solid planar electrode of similar overall dimensions. Additionally, conductive polymers may provide a compliant material interface such that inflammation resultant from mechanical shearing or cellular disruption may be reduced as compared to that arising with electrodes made from relatively non-conforming materials. In addition, conductive polymers may offer an improved efficiency of electrical current transfer at the first surface by having this relatively large surface area and therefore may subsequently enable a smaller overall electrode area as compared to a solid planar electrode. Such polymer electrodes may be constructed in a variety of means, e.g. as conductive materials constructed from fibrous, nanotubes, or nanowires or materials electro-spun or electrochemically polymerized.

In certain embodiments, the electrode as well as the structure supporting the electrode may have features promoting biocompatibility. For example, device surfaces in direct contact with blood may incorporate one or more features such as the addition of materials, e.g. heparin, as well as a smooth topology to limit possible adhesion of blood components and/or disruption of blood flow that in turn may lead to adverse reactions, e.g. clotting. Alternatively, device surfaces in contact with tissues or cells in tissues may have topologies, e.g. grooves or microstructures, and/or added materials intended to minimize adverse body reaction such as fibrous encapsulation or inflammation arising from mechanical shear forces. Other forms and materials for promoting biocompatibility of device surfaces are conceivable and the scope of the present invention is not limited to these examples presented herein.

In various forms of the invention, a first or second electrode, possibly having additional features to guide current flow, may be directly affixed to targeted body structures, e.g. blood vessel walls, by mechanical means or bonded by chemical means. Mechanical means may include circumferential or partially circumferential electrode structures that provide mechanical adherence of one or more electrodes to a target vessel region through compressive force about the vessel. Likewise, sutures, clips or other related structures may be employed to physically anchor electrodes at desired location. Chemical means, e.g. adhesives or biological ingrowth promoting materials, may also be employed to secure attachment to targeted region or vessel.

Attachment of an electrode assembly to a vascular tissue from an outside aspect of the vessel may be challenging in certain applications due to the highly compliant structure of arteries and veins. In such embodiments, electrodes and associated structures are configured to neither adversely constrain a desirable expansion of the vessel nor be so loose fitting that adequate first surface electrical contact is not achieved with the target region. That is, the diameter of a vessel may change over time, e.g. as a vein arterializes, it may double in diameter, or as a vessel changes in dimension periodically, e.g. due to the pulsatile nature of blood flow and the compliance of the vessel and accordingly, a structure of the present invention may be so designed as to conform to these vessel diameter changes.

Accordingly, the present invention may utilize a variety of means to achieve a level of compliant attachment between electrode and its supporting structure and a vessel. In preferred forms of the invention, a structure comprised of a compliant polymer, e.g. silicone, having a plurality of electrodes arranged in a regular pattern is employed. In use, such preferred structures are wrapped around a vessel at a desired location effectively forming a perivascular structure with minimal space between the vessel and the structure. Such structures may be affixed in to position by means of one or more ties wrapped about the structure or by adhesives. Alternatively, flexible meshes having one or more electrodes positioned therein may be employed, e.g. the mesh encircling the vessel then secured in place using ties or sutures.

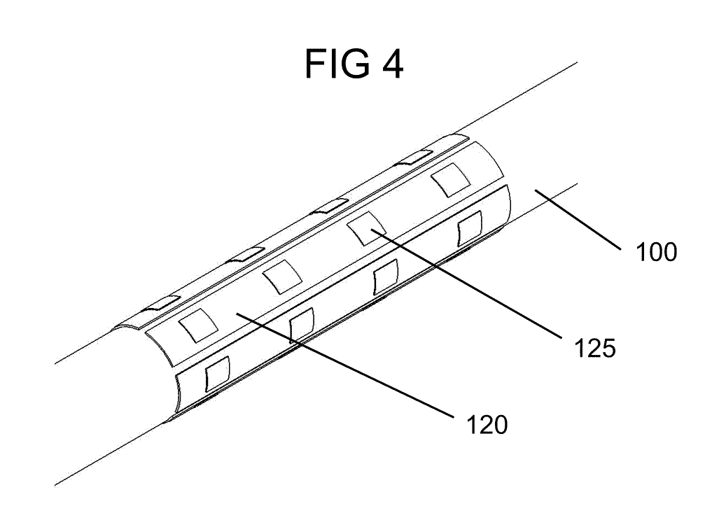

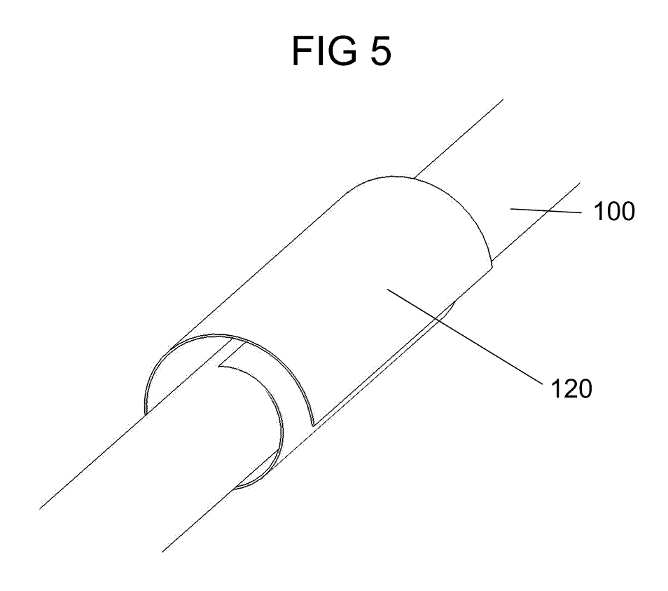

A variety of structures having one or more electrodes positioned or contained within are conceivable in order to promote effective contact of an electrode first surface to a desired vessel tissue or blood vessel region. For example, electrodes may be configured in a variety of forms or arrangements supported in a structure, e.g. matrixes of circular electrodes, partially circumferential electrode bands, electrodes arranged longitudinally in line with the underlying vessel. For example, FIG. 4 presents an illustration of a structure 120 having a plurality of electrodes 125 axially arranged about vascular structure 100. As shown, the structure segments 120 do not circumferentially constrain the radial expansion and/or contraction of the underlying vessel. Not shown in this illustration are necessary electrical connectors, e.g. wires, to the electrodes or additional materials that assist in affixing the electrodes and structures to the underlying vessel, e.g. a stretchable fabric positioned between the supporting structures. As alternative embodiment, FIG. 5 presents an illustration of a spiral cuff structure 120 which is self-sizing and therefore serves to provide dynamic compliance with an underlying vascular structure. In such structures, one may readily conceive of electrodes positioned therein in a variety of forms, e.g. matrixes of circular electrodes, parallel bands of electrodes, etc., according to the current densities and intended use of the device.

Other forms of the invention may include the use of compliant structures which can serve as the scaffold upon which electrode elements are affixed. These structures may make use of specific material types such as absorbable polymers which may dissolve after a set period, e.g. weeks or months, leaving electrodes and associated connecting wires effectively affixed to the vessel wall by the body's foreign body response. The scaffold structures may also include hydrogels which are able to mimic the overall compliance of human tissue. Biological compatible felts, non-woven polymers, foams, knits, and meshes are potential forms of other biocompatible implantable structures that may be used to secure the electrodes in desired locations.

In other embodiments of the present invention, it may be desirable to control or direct current flow from electrode first surface by use of materials having high electrical resistance being in substantial contact with at least a portion of the electrode. Such high electrical resistance materials may preferentially direct current flow from the electrode into a vessel wall or other target region and minimize non-useful electrical paths through surrounding body tissues. In various embodiments, electrical isolation may be achieved through use of insulating structures that most nearly match the compliance of the electrode structures. Compliant, insulating structures can be made from a host of commonly available materials including low durometer silicones, closed cell foams, non-conductive hydrogels, etc.

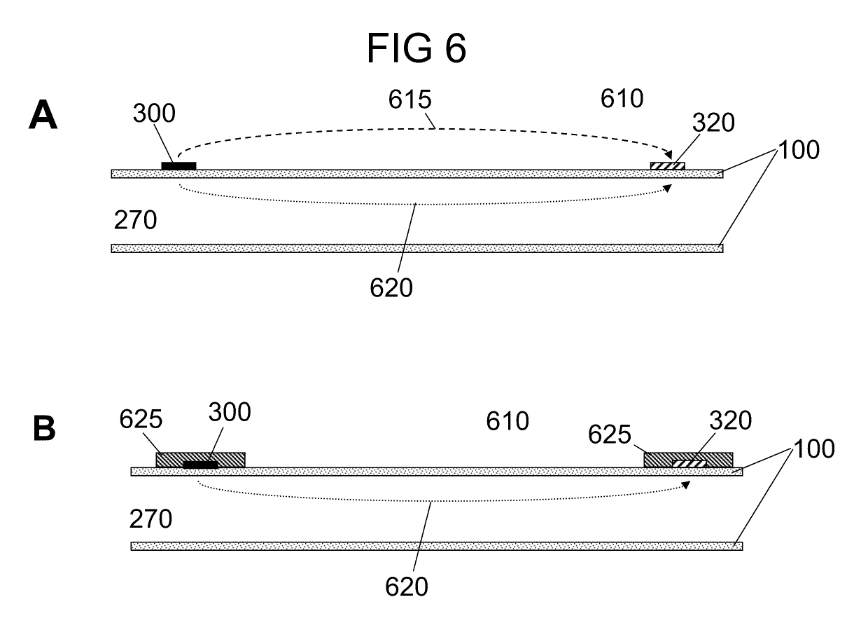

An example illustrating the basis for this need for electrical isolation is presented in FIG. 6. As shown in this side view, blood vessel 100 defining luminal space 270 that is filled with blood, an electrically conductive body fluid. Also shown is surrounding body tissue 610. Upon activation, an electric current is passed between a first electrode 300 to second electrode 320. Panel 6A illustrates the condition wherein a portion of the electrical current 615 non-desirably traverses tissue 610. Another portion of the electrical current 620 passes desirably through vessel wall 100 and luminal space 70. Panel 6B illustrates the advantageous use of high resistive materials 625 positioned about electrodes 300, 320 that serves to preferentially guide electrical current passage to desirable directions. In general terms, the electrical path is typically the pathway offering the least overall resistance to electrical current passage. By positioning high resistive materials 625 such that the path of least resistance is that offered through vessel wall 100 and luminal space 270 then as indicated by arrow 620, the preferential route of electrical current with minimal current loss to non-desired pathways is through vessel wall 100 and luminal space 270.

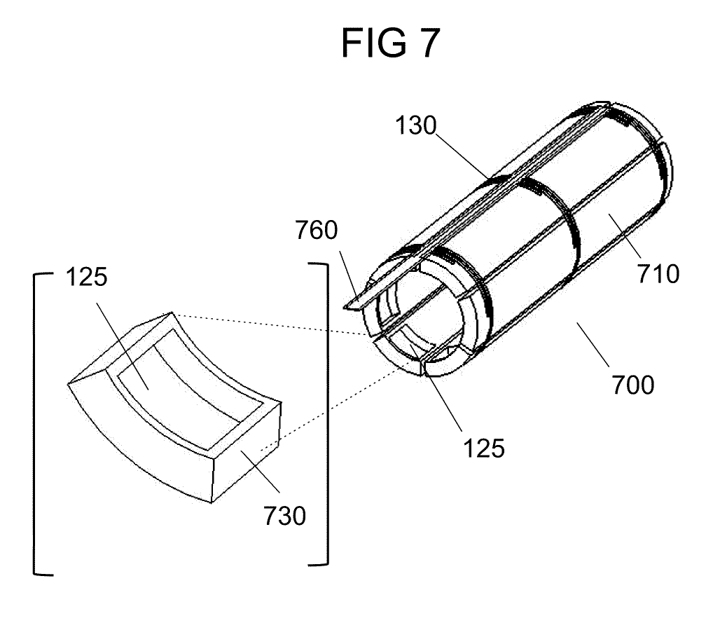

An illustration of how insulating structures may be employed with conductive polymers or gels utilized as electrodes is shown in FIG. 7. As shown, structure 700 is configured to enable placement about a vessel. Structure 700 contains separated electrode support elements 710 to enable flexure/expansion of the vasculature. Also shown are electrical connections (traces) 130 to enable electrical connection between electrode 125 and electrical connection 760 that further enables electrical connection to a controlling circuit module (not shown). To better enable the employment of conductive gels or other conductive polymeric materials as electrodes, open box-like structures 730 are arranged within support 710 wherein said boxes may be highly conformable, e.g. comprised of silicones or other such materials, as well as highly resistive, thereby resulting in a preferred electrical path through tissues and body fluids from electrode 125 to other electrodes 125 positioned in overall structure 700.

In yet other forms of the invention, compliance of the structure in contact with the vessel may be enhanced by spacing elements with lesser compliance or resilience, e.g. metal electrode first surfaces, from direct contact with the vasculature tissues. For example, non-conductive foam-like structures saturate-able with conductive biofluids may be interposed between the target tissue and non-resilient electrode structures such as wires, foil, or other electrically conductive metallic structures. A variety of materials may be utilized in such interposition fashion, including hydrogels or open-celled foams. Furthermore, this intervening volume may be filled with a combination of conductive and non-conductive compliant elements. In such embodiments, a second structure may be utilized to organize the interposing material and maintain at a predetermined distance between electrode and the target tissue, blood or graft. That is, to facilitate assembly and placement, a mechanical structure, e.g. knitted mesh, non-woven, felt, foam or other stretchable material, may be utilized to wrap or otherwise contain the electrode assembly and intervening foams, etc.

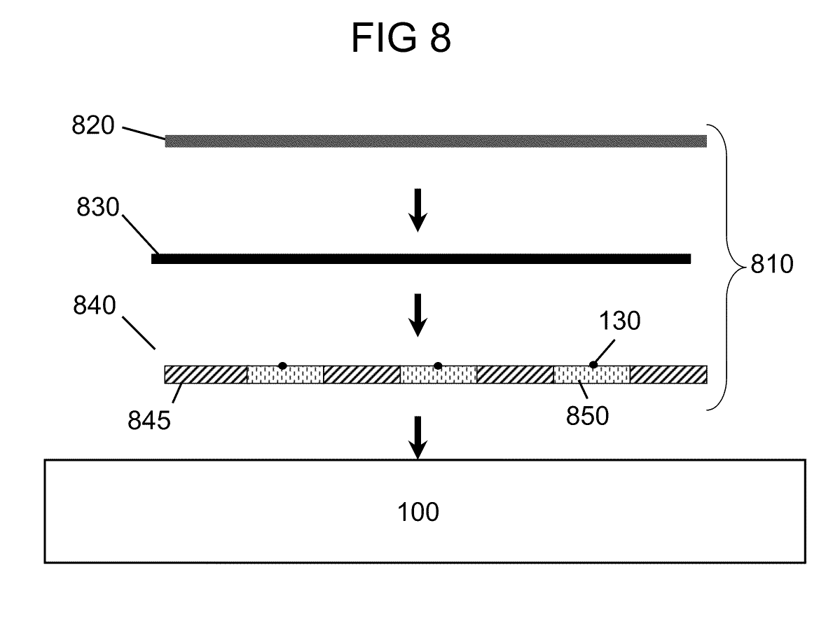

An illustration of one form of this embodiment of the invention is shown in FIG. 8. As shown in this side view, electrode structure 810 is comprised of stretchable wrap 820, insulating layer 830, and gel layer 840. Gel layer 840 in turn is comprised of insulating hydrogel sections 845 with conductive hydrogel sections 850 interposed. Electrical contact to conductive hydrogel sections 850 is through metallic wires 130. Overall structure 810 is intended to be placed in substantial contact with blood vessel 100 to enable the present invention, as indicated by solid arrows.

In various embodiments, one or more support structures may be constructed in whole or in part from naturally occurring polymers to which electrically conductive elements, e.g. electrodes, are affixed. Such naturally occurring supports may then offer more natural resilience and improved biocompatibility as compared to synthetic materials. In addition, surrounding tissue in-growth and acceptance may be improved by such materials. Such naturally occurring materials may or may not have cellular components contained within the material to aid in device performance, longevity and/or tolerance by the recipient. In certain embodiments, a target region of the vasculature may be constructed in part or in total from a patch or graft comprised of such naturally occurring matrixes or materials previously prepared with one or more electrode elements.

Another possible form for constructing electrodes and/or supporting structures involves the use of materials that are injected as liquids into a desired region which then are cast or set to a desired shape in situ using a mold placed around the target vessel or graft. Alternatively, the casting material may be delivered as a bolus of material which then sets or forms about at least a portion of a target vessel or graft without the use of a mold. Such embodiments, either with or without the use of molds, may enable improved compliance on the part of the material, e.g. hydrogel. Advantageous use of such embodiments may arise from improved compliance more closely matching that of the target tissues/grafts. Such embodiments may have the added benefit of taking the exact form needed for ensuring close contact to the target vasculature or graft.

That is, forms of the invention utilizes liquid materials which then assume a solid dimension in situ may be well suited to applications having highly irregular or different dimensionalities in body vasculature as well as to possible the dynamic changes of the vasculature, such as the unique shape and remodeling of blood vessels associated with arteriovenous fistula remodeling. One embodiment of this form of the invention may involve the injection of conductive hydrogel electrodes into pre-determined cast forms (rings, bands, spots, etc.) surrounding the vessel at key locations useful for the delivery of electrical energies. These conductive elements may in turn be connected to an electrical current source via conducting metal wires or similar conductive elements that are insulated to prevent unwanted current paths.

A non-conductive injectable polymer may then be used to fill in all of the spaces surrounding the electrodes/conductive hydrogel. As one variant of this form of the invention, foams constructed in part from naturally occurring materials, e.g. collagen, also having polymeric expansion agents and/or other added materials such as conductive polymers may be employed to provide either conductive or non-conductive materials in situ, as needed. In general terms, biocompatible polymers that are curable or moldable in body may include forms of silicones, polyurethanes or other elastomeric substances that when exposed to the appropriate curing agent, e.g. ultraviolet light, or the presence of oxygen, which then initiates polymerization into a non-flowing form.

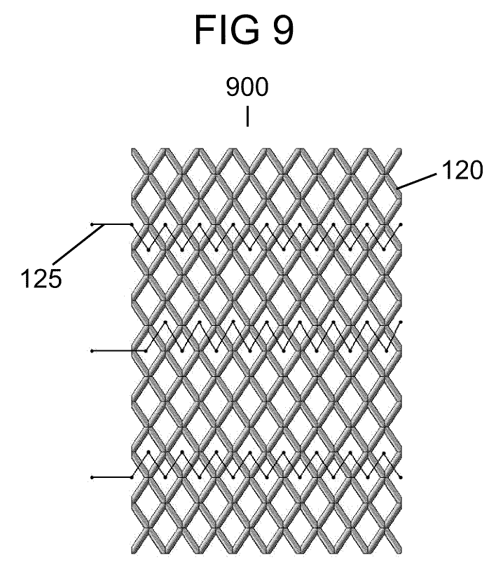

In yet other embodiments, compliant structures, e.g. meshes, fabrics, foams, having conductive elements that serve as electrodes may be applied directly to the target vasculature. These constructs may be fabricated from medical grade non-conductive materials interlaced or woven with conductive strands or elements that thus form the conductive electrode elements. In the case of wire strands, these elements may be woven in a serpentine fashion to provide strain relief in those directions where highest compliance is needed. An example of such a form of the invention is shown in FIG. 9. As shown, overall structure 900 is planar in form and comprised of mesh-like support 120 with electrically conductive wire electrode 125 intertwined. Not shown is an insulating layer to be placed on one surface of planar structure 900 which would serve to prevent electrical conduction through tissue and body fluids in contact with this surface.

In alternate embodiments, electrodes of the present invention may be contained within tubular structures that are utilized to convey body fluids such as blood, e.g. synthetic grafts, or within other medical or implanted devices and thereby be utilized to mitigate the body's response to these structures. For example, a synthetic graft constructed of expanded polytetrafluoroethylene (ePTFE) may have one or more electrodes so positioned within the graft such that a first surface of the electrode is in effectively direct contact with the blood and thereby may be utilized to affect body responses in the immediate region. In general terms, such tubular structures (or other devices) may be comprised of a variety of materials, ranging from compliant, electrically resistive polymers such as ePTFE, to more rigid conduits comprised of harder plastics or metals.

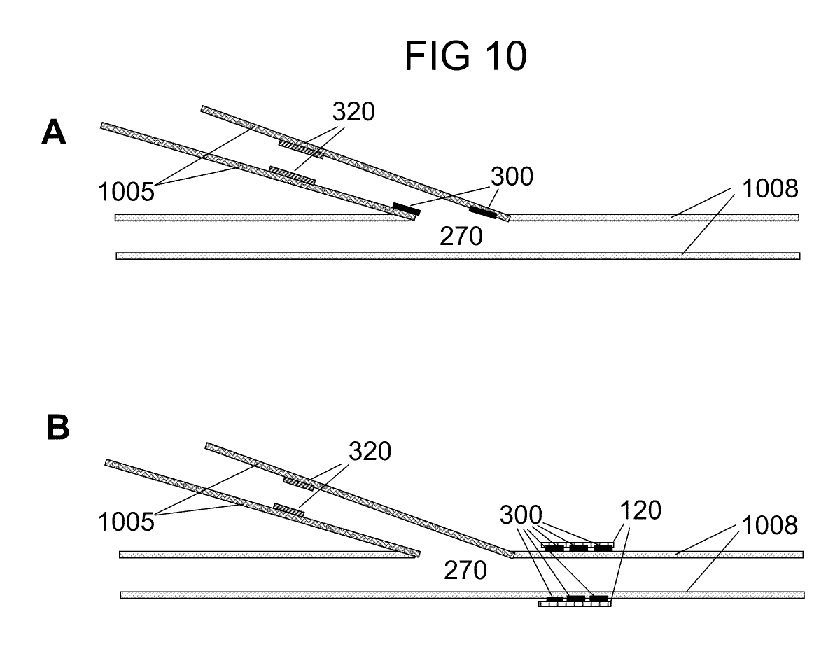

The preceding focuses on illustrative uses of the present invention intended for placement about a vessel. Alternate embodiments of the present invention may be incorporated in medical structures such as grafts which serve contain or guide blood passage between naturally occurring vessels. For example, FIG. 10 illustrates use of elements of the device, e.g. electrodes, contained within the luminal aspect of the graft thereby coming into direct contact with vascular blood flow. As shown in Panel 10A, first electrodes 300 may be located within graft 1005 in the vicinity of the anastomosis or junction between graft 1005 and blood vessel 100 such that electrodes 300 may be employed to manage patency at the anastomosis. Second electrodes 320 are positioned within the graft and have dimensionality intended to preclude effective electric field strength from being experienced in their vicinity. An alternative embodiment is shown in Panel 10B, where first electrodes 300 may be affixed in flexible mesh structure 120 positioned on outside of the adjacent blood vessel with second electrodes 320 positioned within graft structure 1005. Activation of first electrodes 300 is intended to mitigate neointimal growth or other undesired vascular responses in this vascular region by applying the electric current through the blood vessel wall to electrodes 320 with the current flow necessarily restricted to vascular lumen 270 due to the high resistivity of graft 1005. In a further refinement of this embodiment, the series of concentric first electrodes may be sequentially activated, thereby enabling further control of patency in this vascular region.

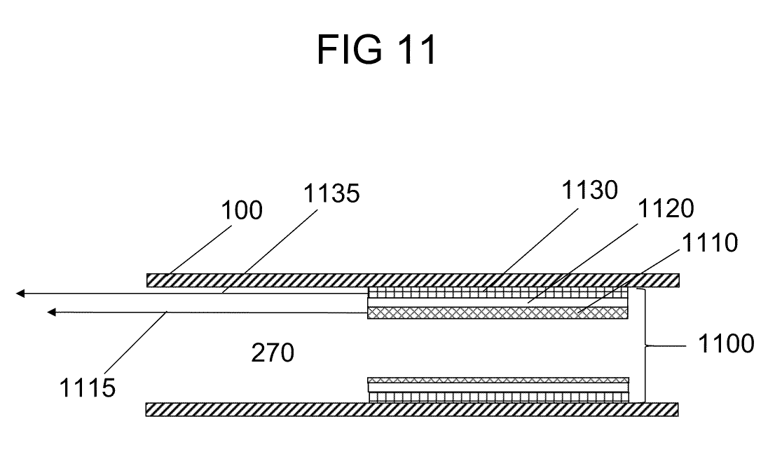

Yet other embodiments of the present invention incorporate elements of the present invention in devices positioned in the vascular lumen. For example, FIG. 11 illustrates an implantable vascular device having two concentric stent electrodes radially-separated by a semipermeable layer. The device comprises of an expandable tube 1100 having three concentric and radially-separated sections: an inner stent 1110, an insulating layer 1120, and an outer stent 1130 positioned on the inner aspect of vascular structure 100. In the preferred embodiment, the inner stent 1110 and the outer stent 1130 are formed of an expandable metal wire mesh tube; and the intermediate insulating layer 1120 is formed of an expandable plastic mesh tube. Layer 1120 physically separates the inner stent 1110 from the outer stent 1130, thereby requiring current flow to proceed through body fluids or tissues in this region. The device further includes a first connection wire 1115 and a second connection wire 1135 and a power supply/control unit (not shown) with power supply/control unit having a first output and a second output.

In this embodiment, inner stent 1110 forms a first electrode of the implantable vascular device and is electrically connected to one end of the first connection wire 1115. Likewise, the second-electrode section 1130 forms the second electrode of the implantable vascular device and is electrically connected to one end of a second electrical wire 1135. The other end of the second electrical wire 1135 is connected to the second output of the power supply/control unit. In one embodiment of this form of the invention, the first electrode 1110 may be biased to serve as an anode.

In operation, the expandable tube 1100 is placed in a desired vascular location, e.g. where a constriction in the blood vessel exists. The power supply/control unit delivers a pulsatile direct current (DC) signal between the inner stent electrode 1110 and the outer stent electrode 1130, thereby resulting in an electric current to flow between the electrodes. In this embodiment, a majority of electric current is intended to flow along the radial direction towards the outer stent electrode 1130 and is intended to influence the movement of cells and materials responsible for neointima, i.e. reduce the density of neointima in the critical inner vascular region 70. The intended result is a reduction of the rate of restenosis (re-clogging) in the critical blood-flowing region of the inner luminal space.

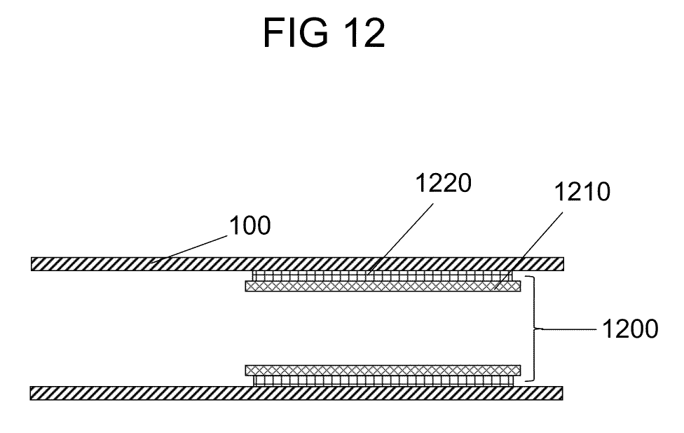

In a yet other form of device incorporated into a form of stent is illustrated in FIG. 12. As shown, this form of an implantable vascular device is comprised of an expandable tube 1200 having two concentric and radially-separated stents: an inner stent 1210 and an outer stent 1220 positioned in vessel 100.

In the preferred embodiment, the inner stent 1210 forms a first electrode and the outer stent 1220 forms a second electrode. Inner stent 1210 may be formed of a first expandable wire metal wire mesh tube and outer stent 1220 may be formed of a second expandable metal wire mesh tube wherein the metal forming the inner stent 1210 differs from the metal forming the outer stent 1220, resulting in a galvanic potential difference and current flow through lumen space without requiring an external power supply/control unit. Return current flow between stent 1210 and stent 1220 is achieved by an electrical connector, e.g. a wire (not shown). This configuration may direct the flow of healing cells in a preferential manner for a period of time long enough to achieve the desired therapeutic outcome, prior to a critical loss, e.g. galvanic corrosion, of the stent serving as the anode.

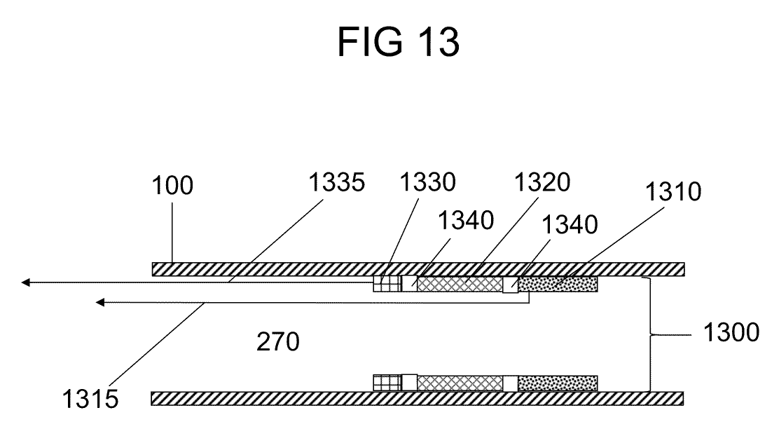

FIG. 13 illustrates yet another an implantable vascular device, this being comprised of axially separated stent-like electrodes. As shown, the device is comprised of an expandable tube 1300 having three separate sections: a first electrode section 1310, a spacer section 1320, and a second-electrode section 1330. In the preferred embodiment, all three tube sections are comprised from an expandable metal wire mesh. The expandable tube 1300 positioned in vessel 100 also includes two insulating rings 1340 that prevent direct electrical contact between tube sections 1310, 1320, and 1330. The device further includes a first connection wire 1315 and a second connection wire 1335 and a power supply/control unit (not shown).

In use, the first electrode section 1310 may be positioned at a target vessel region, e.g. where a stenotic constriction in the blood vessel exists or is predicted. Section 1310 therefore serves as the first electrode of the device 1300. Second electrode section 1330 may then be positioned at a distance from electrode 1310, where this distance may be governed by the length of the spacer section 1320. First electrode 1310 is electrically connected to one end of the first connection wire 1315. Connection wire 1315 is electrically connected to a first output of the power supply/control unit (not shown). Likewise, the second-electrode section 1330 is electrically connected to second electrical wire 1335. The other end of the second electrical wire 1335 is connected to a second output of the power supply/control unit (not shown).

In operation, the power supply/control unit may deliver a pulsatile DC signal between the first electrode 1310 and the second electrode 1330, thereby resulting in electrical current flow between the electrodes. Given the particular arrangement of electrodes in this preferred embodiment, a majority of electrical current will flow along the axial direction towards the second electrode 1330. The electrical current results in electric fields intended to reduce the density of neointima in the critical inner region of the expandable tube, e.g. the inner luminal blood flow region 70. Neointimal formation may arise at the outer electrode instead of the critical inner region. The result may be a reduction of the rate of restenosis (re-clogging) in the critical blood-flowing region and therefore longer useful patency.

In an alternate form of this embodiment, section 1310 and the second section 1330 may be separated by a single plastic mesh tube, replacing the metallic spacer section 1320 and the insulating rings 1340. In yet another alternate embodiment, the second electrode section 1330 is separate, mechanically as well as electrically, from section 1310, although both sections may be deployed at the same time. This may be accomplished by spacing the sections suitably apart on an insertion rod before introducing them into the blood vessel and expanding them, thereby fixing these in place in the lumen. Yet in another alternate embodiment, to mitigate possible detrimental effects within a reactive electrode electrolysis zone, one or more electrode first surfaces may be coated or covered with a semipermeable mesh to separate the electrodes from the surrounding tissues. One means to accomplish this is shown in FIG. 2 where the electrode section is encapsulated within outer and inner semipermeable layers.

In yet other embodiments, a plurality of first and second electrodes are utilized. This may be achieved by a series of the three-sectioned tube structures all connected along the length-direction, with each structure having the first-electrode/spacer/second-electrode arrangements. By sequential activation of sets of these electrodes, e.g. in a wave-like pattern, non-desired cells or biological responses maybe directed away from the critical section towards non-critical locations of the implanted vascular device.

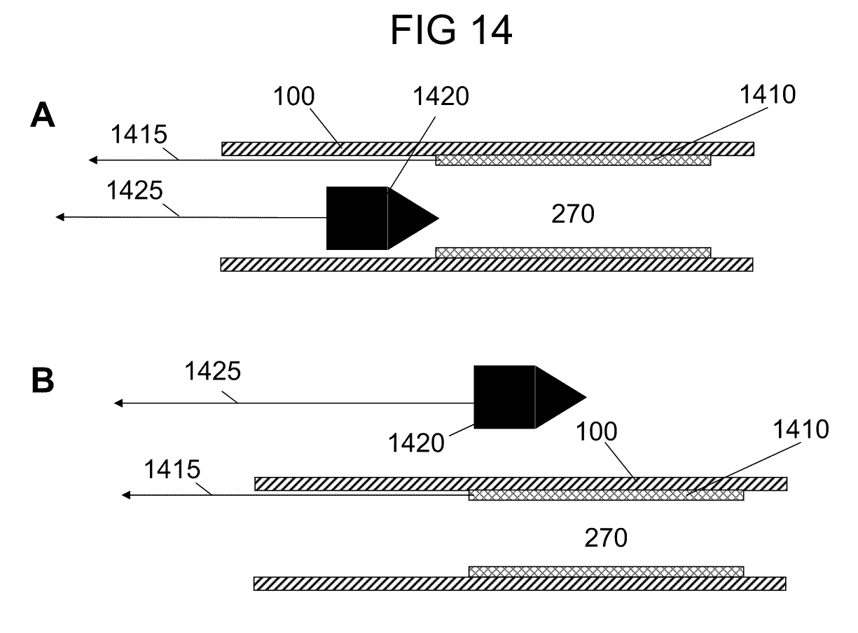

FIG. 14 illustrates still another form of an implantable vascular device of the present invention. As shown in Panel 14A, the device is comprised of a stent electrode 1410 configured to be placed inside the lumen 270 of blood vessel 100 and a non-stent electrode 1420 also configured to be placed inside the lumen of blood vessel 100. In one form of this embodiment, stent electrode 1410 may be comprised of wire mesh tube and forms the first electrode. Non-stent electrode 1420 forms the second-electrode and may be independently introduced into the blood vessel at a fixed distance from the stent-electrode 1410. The stent electrode 1410 is connected to a first output of the power supply/control unit through a first connection wire 1415. Likewise, the non-stent electrode 1420 is connected to a second output of the power supply/control unit through connection wire 1425.

The operation of this embodiment is substantially similar to that of the implantable vascular device described with respect to FIG. 13. The power supply/control unit may deliver an electrical energy, e.g. a pulsatile unidirectional current, that passes between stent electrode 1410 and non-stent electrode 1420. The resultant electrical current passes substantially along the axial direction away from the stent electrode 1410.

FIG. 14B illustrates a related embodiment wherein stent electrode 1410 is positioned inside the blood vessel 100 and a non-stent electrode is positioned outside the blood vessel 100. The stent electrode 1410 forms the first electrode and is typically made of wire mesh tube. The non-stent electrode 1420 forms the second-electrode. Electrode 1420 may be independently introduced into the body and positioned as compared to electrode 1410. It will be readily recognized that placement of second electrode 1420 outside the blood vessel will result in electrical current flow through the blood vessel wall, thereby resulting in electric fields to be oriented across the wall of the blood vessel and encouraging movement of non-desired cell types from luminal regions to the outer aspects of the vessel wall. Such configurations necessarily require some form of electrical connection traversing vessel wall in order to complete the circuit, utilizing power and signals from a power supply/control unit (not shown) located either within the vessel itself or in surrounding tissue.

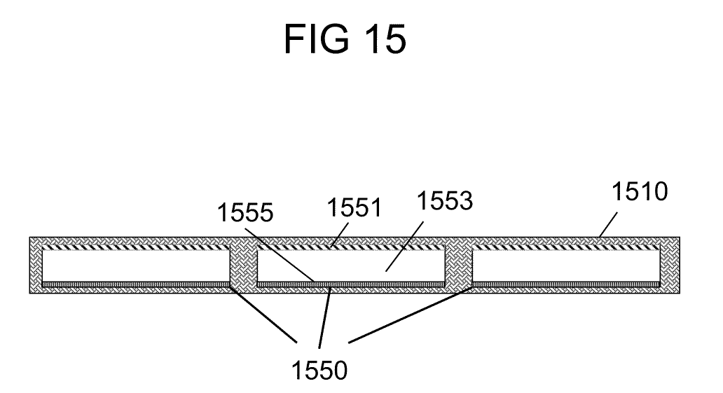

FIG. 15 presents a cross section of a stent-like structure with incorporated capacitor elements according to an embodiment of the present invention. Stent structure 1510 has capacitor elements 1550 located within. Each of the capacitor elements 1550 has an upper plate 1551, a dielectric layer 1553, and a lower plate 1555. In the preferred embodiment, these capacitor elements are charged externally prior to the implantation of the stents. In an alternative embodiment, these capacitor elements may be connected to and driven by to a power supply/control unit.

In operation, each of these capacitor elements upon discharge generates a local electrical current that may guide neointima forming cells towards the upper plate 1551, thereby reducing the neointima formation in the critical inner region of the stents.

Activation of electrodes for the purpose of the passage of an electric current through target tissue or body fluids may be accomplished in a variety of fashions, including, but not limited to, activation upon command, activation periodically, being activated substantially continuous fashion, e.g. always “on” or variable activation based on a schedule or patency status. An example of variable activation is the employment of frequent pulses of electrical current immediate following post implantation of the device, e.g. 24-72 hours, intended to manage the immediate local tissue reaction to implantation surgery and the cell types associated with this response. This may then be followed by an altered regimen, e.g. a reduced application periodicity, for the remainder of the implant's useful lifetime that may be suited for chronic vascular remodeling activity entailing different cell types.

In alternate embodiments of the invention, activation of one or more sets of electrodes may coincide with a therapeutic activity or in response to sensor input. In select embodiments, the therapeutic activity may be a hemodialysis session wherein the electric field activation coincides with the dialysis session. In a further refinement of this embodiment, one or more of the dialysis needles may serve as either a first or second electrode. Accordingly, a variety of activation schemes and profiles are possible within the scope of this invention and this invention is not limited to the embodiments described.

One embodiment of the present invention may use of multiple electrodes in the form of an array. Such designs may employ use of technologies such as micro-wired connections, printed transistors, flexible circuitry, conductive polymers, micro-wells, etc., to achieve an electrode array structure with the ability to produce a variety of electrode activation arrangements. That is, selective activation, including selecting polarity, of one or more electrodes may be utilized to alter or affect the effective electrode size and location. Such matrixes enable the ability to specifically target vascular tissue regions at intended times while allowing other regions remain untargeted. Such capabilities enable a progressive targeting of one or more tissue regions in contact with the electrode matrix and thereby advantageously conserve peak power requirements of the device, i.e. as compared to fully activating the matrix as a whole. Such a matrix electrode structure is illustrated in FIG. 1.

Overall, a variety of first and second electrode shapes, form and materials as well as supports are conceivable and the scope of the present invention is not restricted to any one type, shape or form of these.

Control and power for the delivery of an electrical current signal may be accomplished with circuitry as simple as a battery plus microcontroller or as complicated as an external power circuit plugged into a wall plug plus controlling software being remotely linked to the implanted system. In this latter scenario, power may be supplied using inductive or other power coupling means to provide energy to implanted batteries or other forms of power storage.

Power may also be supplied by other sources of energy. For example, a variety of technologies exist which convert mechanical movements into electrical energy. In select instances, perivascular structures may be employed for such energy generation purposes. One embodiment of such a perivascular device may include a compliant polymer (e.g., silicone) sheet impregnated with ultra-thin piezo electric elements such that when device is flexed, i.e. during pulsatile blood flow, electricity is generated which then may be used for powering a least a portion of the present invention. In various forms, such devices might consist of ceramic nanoribbons (or other piezo electric materials) embedded onto silicone rubber sheets, which can generate electricity when flexed thereby converting mechanical energy into electrical energy.

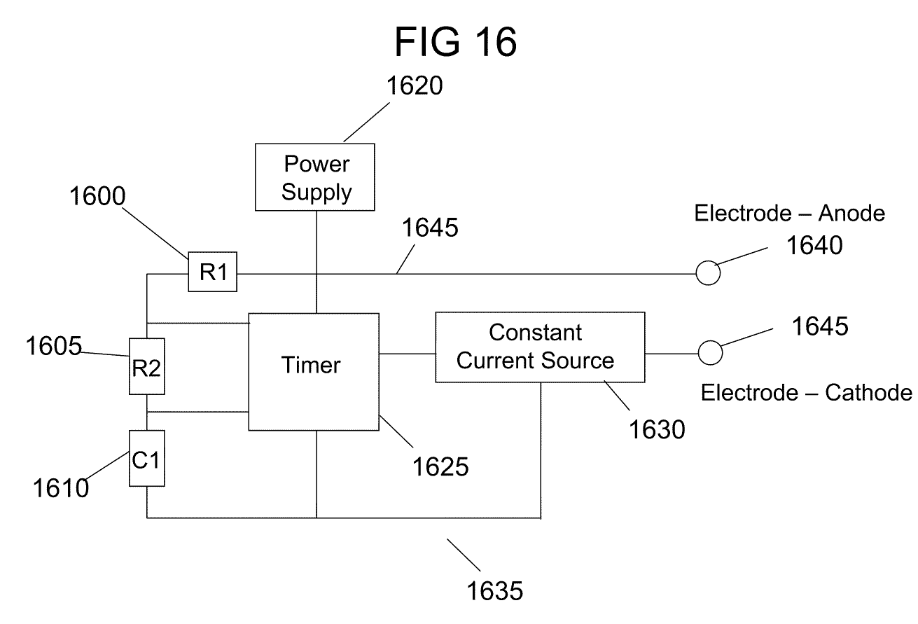

FIG. 16 illustrates the components of one circuit for the controlled delivery of pulsatile DC currents to electrodes. One skilled in the art of electronics will readily recognize that numerous other circuits that accomplish this purpose are conceivable and are covered within the scope of this invention. Power is supplied by the power supply 1620, typically a battery connected by wires 1645 to circuitry 1635. The repetitive pulse is generated within the timer 1625 e.g. an integrated circuit available from Texas Instruments, Philips Electronics, National Semiconductor, etc. Frequency and duty cycle are determined by external resistors, 1600, 1605 and capacitor, 1610. The output of the timer drives a constant current source which in turn, provides the constant current source 1630 through the circuitry 1635 to the anode electrode 1640 and current sink to the cathode electrode 1645.

An example calculation for determining duty cycle employing the circuitry of FIG. 16 is shown in Equation 1:

Duty cycle (Ratio of ON time to OFF time)=R2/(R1+2R2) Equation 1:

Assuming R1=98 kohm and R2=1 kohm and C=10 uf, then the duty cycle equals 1/(98+2*1) or 1% and the pulse frequency equals 1.44 Hz. One skilled in the art of electronics will readily appreciate that more complex circuits, involving delays, changes of pulse amplitudes or frequencies as well as additional variety of pulse patterns may be readily conceived and employed within the scope of this invention.

In one embodiment of the invention, regulation of the control circuitry, e.g. the programming of the amplitude and periodicity of the current to be delivered, is set prior to installation of the invention into a medical device. In another embodiment of the invention, a separate means to adjust or provide control electrical current output post-installation is provided. Such means include, but are not limited to, keypad entry, wireless control, or by optical or acoustic means. In addition, a variety of means may be employed to turn device on/off, including magnetic switches, automatic activation upon contact with body fluids, etc.

Other embodiments of the invention providing for adjustment/activation of the currents applied also include the use of input or controls provided within a larger medical device or system employing this invention. In yet other embodiments of the invention, feedback from sensors indicating the need to alter the current profile, either associated with the apparatus of this invention or as part of other devices, may be sent to a control circuit in an automatic fashion and thereby providing a “closed-loop” system of operation of the apparatus of this invention within the body of a subject.

The introduction of an electric current into fluid such as blood may result in several possibly detrimental side effects, dependent upon the nature and extent of applied current and the dimensions/types of electroactive surfaces, e.g. electrodes, employed. These effects may include the generation of acid and base at the anode and cathode (respectively); the formation of a highly reactive electrolysis zone immediately adjacent to the electrode surface; and the possible formation of gas bubbles at the electrodes. One possible means to reduce or minimize possibly detrimental activities is to introduce the current in a modulated, e.g. pulsatile, fashion, analogous to the passage of high frequency electrical signals through capacitors. By doing such, possible Faradaic chemical reactions at the electrode surface are minimized, lessening the generation of the deleterious agents.

Modulated currents are typically characterized by the pulse amplitude, pulse frequency and the on/off percentage of time during the pulse frequency period (otherwise known as the duty cycle). In addition, the composition and viscosity of the surrounding electrolyte fluid, e.g. body fluids such as interstitial fluid, cerebrospinal fluid, etc., as well as the electrode material and current density influence the nature and extent of the formation of electrolysis by-products.

In a preferred embodiment of the invention, pulsatile DC currents are utilized to minimize possible deleterious products and reduce power consumption. In this embodiment of the invention, the pulse frequency is generally between 0.1 Hz and 1000 Hz, the duty cycle is generally between 0.1% and 10% and the current density is generally between 0.01 mA/cm2 and 100 mA/cm2 at first electrode surfaces. However, the broader scope of this invention is not intended to be limited by this embodiment and conditions. It is noted that other conditions, materials and structures may be employed such as those described by in the following sections that permit wider current limits and parameters, including continuous application of direct current and/or use of random or semi-random electric current applications.

In other forms of the invention, a substantially continuous current may be employed. In still other forms of the invention, the polarity of the current may be reversed periodically or on command, e.g. to minimize Faradaic effects and/or to maintain the structure/integrity of one or more electrode surfaces, if needed.

The introduction of the electric current into a conductive medium, e.g. interstitial fluid or blood, may result in the electrolysis of water, forming either acid or base in the vicinity of the electrode (typically acid, e.g. hydronium ion H+, at the anode and base, e.g. hydroxide anion OH—, at the cathode). In certain situations, the generated base or acid may overwhelm the surrounding medium's buffering capacity, substantially altering the local pH and potentially adversely affecting the surrounding tissues and cells. One embodiment to ameliorate this generation of acid or base is to employ a modified form of electric current delivery whereby the polarity of the electrodes is reversed periodically. That is, although the electric current application may be substantially DC in nature; by altering the polarity of the electrodes intermittently, an electrode which had been the site of acid generation now becomes a source of base generation, and vice versa. This switching of polarity, if performed with the appropriate periodicity, may substantially eliminate adverse pH effects yet may have minimal effects upon the net migration of the targeted cell types, etc. That is, the polarity reversal is for such a short period that major drifts, cell processes or motions are not substantially reversed. In one embodiment of this invention, the polarity is reversed in an asymmetric fashion, such as by time of pulse period or by current amplitude, to achieve neutralization of generated acids or bases.

An alternate embodiment of the invention may be to provide additional buffering materials or compounds either as part of the structure or as delivered solutions. That is, the structure of the device may be composed of materials which function in part as a binder to the acid/base such that the acid or base generated is immediately bound to the material, thereby neutralizing these reactive species. Such materials may include structural carbonates or coatings of ion exchange resins. This method may be used alone or in combination with the alternating polarity mentioned above to negate the effects of generated acid or base.

The process of electrolysis or breaking down of water molecules may create a highly reactive zone of chemical species extending from the surface of the electrode into the surrounding tissues or fluids, up to several hundred nanometers, dependent upon, among other factors, the structure and composition of the electrode, and the electrode potential applied. This zone may be harmful to the surrounding tissue directly or the process of electrolysis and the agents generated may induce a rejection response in the region, e.g. through the formation of radicals which generate antigenic species. In one form of the invention, the electrodes may have electrically active surfaces positioned away from the surrounding tissue at a sufficient distance to mitigate the effects of electrolysis, e.g. a distance generally greater than 1 micron, and thereby segregating the tissue from this highly destructive environment.

Accordingly, in one embodiment of this form of the invention, the first surface, i.e. active surface, of electrodes is physically separated from the tissue by an overlying semi-permeable structure or gel. A semi-permeable structure in the context of this invention may be a structure, membrane, mesh or gel, which provides fluid and small molecule access to the electrode surface while physically distancing the electrode from contact with surrounding tissue. Therefore the dimensionality of the pores of such a structure is preferably less than the dimensionality of the surrounding cells and tissues. In general, a pore size that is less than 5 microns in diameter is desirable, and less than 1 micron more desirable, to prevent cellular infiltration. In alternate embodiments of the invention, larger pore dimensions may be employed wherein the overall fluid path length or tortuosity is increased. Such approaches thereby may permit the use of meshes or polymers with pore sizes considerably larger in diameter, e.g. 1 mm.

Another by-product of electrolysis may be gas generation at one or more electrodes. In aqueous solutions, the positively biased anode typically may generate oxygen while the negatively biased cathode typically may generate hydrogen. The amount of gas generated is dependent upon the current utilized and the electrode employed. If the rate of evolution is sufficiently low per unit area, then the generated gas will dissolve into the surrounding fluid without gaseous bubble formation (this is dependent, among other factors, upon the rate of electrolysis per unit area, electrode composition, surface roughness of the electrode, etc.). However, if higher currents are required in order to minimize the body's rejection response, the overall electrode dimension, shape and number of electrodes may be altered to accommodate higher currents necessary to mobilize the biomolecules while avoiding bubble formation. Therefore, in one embodiment of the invention, gas bubble formation is minimized by enlarging the electrode surface area relative to the current employed in order to facilitate diffusion of the gas into the surrounding fluid. Such enlargement of surface area also may benefit charge transfer characteristics of the electrode, in general.

An alternate embodiment by which to minimize gas bubble formation is to employ agents that absorb the gas as it is generated. This may be accomplished using materials which are employed also as electrodes. This is the case with certain metals, e.g. titanium or platinum at positively biased electrode (anode) which may form oxides in the presence of the generated oxygen or palladium at the negatively biased electrode (cathode) which absorbs hydrogen. Alternatively, these materials may be located near to the electrodes but not necessarily serving as the electrode, e.g. a mesh or structure overlaying the electrode which absorbs the gas in question.

In alternate embodiments of the present invention, other energies may be employed to regulate the proliferation or movement of one or more cell types associated with a desired therapeutic response related to vascular patency. Such energies may include, but are not limited to photonic energies, electromagnetic energies, mechanical, chemical or thermal energies delivered from one or more energy delivery sources. For example, the application of red or near infrared light may result in the desired proliferation of selected cell types, e.g. endothelial cells, involved in achieving desired coverage of injured or disrupted vessel luminal walls, or the reduction of inflammatory responses associated non-desired vessel wall remodeling and/or stenotic lesion formation.

By way of example, the delivery of one or more photonic energies to targeted vascular regions may be accomplished by use of substantially planar light sources. Such light sources include, but are not limited to organic light emitting diodes (OLEDs), light emitting diodes (LEDs) and micro-plasma light sources. It will be readily understood by those skilled in the art of electronics that such light sources, as well as other energy sources, may utilize forms of electrical connections and controls as well as power sources similar in concept to those mentioned in the preferred embodiment of the present invention. In form, such the energy generating component, e.g. a LED light source, may be in direct contact with a target tissue or vessel region and mounted on a structure effectively circumferentially bounding the vessel region. Alternatively, the energy may be conveyed to the desired region by means of a transferring structure, e.g. a fiber optic cable, such that the mass of the energy source does not exert a direct and possibly deleterious impact on the vessel or targeted tissue region.

In addition, a plurality of light sources able to generate one or more wavelengths of light may be arranged in or about a vascular structure or tissue such that targeted delivery of light energies may be made upon command to one or more vascular regions. This will be readily understood that such light sources and targeted delivery may be similar to the targeted delivery of electrical energies from a plurality of electrodes, such as those employed in preferred embodiments of the present invention.

In yet other embodiments, other forms of energy may be utilized in combination with the energies intended to guide cellular processes over extended periods of time. An example of one such form is the controlled intense delivery of acoustic, radiofrequency or thermal energies resulting in cell death of one or more cell types in a targeted vascular region. The scope of additional energies that may be applied in conjunction with one or more the energies of the present invention that are intended to guide cellular processes is not restricted to any one type or form of energy.

To further extend the utility of the invention described herein, additional technologies or methods may be employed to aid in the acceleration or retardation of desired cellular processes and thereby manage patency. Such technologies may include the use of chemicals, biological agents, nanotechnologies, conductive polymers, zwitterionic materials, optical/photonics, acoustical energies, electromagnetic signals including radiowaves, thermal energy, and/or mechanical devices. These technologies may be actively applied, e.g. radiowaves, to affect other aspects of the tissue structure, e.g. disruption of unwanted fibrosity, not readily accomplished by the levels or delivery paradigms employed for the delivery energies of the present invention. Alternatively, incorporation into one or more structures of passive materials, e.g. zwitterionic coatings, may further aid in the desired management of vascular patency.

For example, work by others has observed that magnetically responsive cells may be utilized to improve endothelialization of stents and reduce in-stent restenosis. The magnetic cells may be produced by obtaining autologous endothelial progenitor cells from a blood sample of a patient and loading them with magnetic nanoparticles. In the context of the present invention, guidance to one or more vascular regions may then be accomplished by the controlled application of electromagnetic forces produced by devices of the present invention. Such guidance may extend beyond the report use for stents and may include use in the endothelization of grafts and/or shorten the time of recovery of vascular surgeries such as artervenous fistula formation while concurrently lessening the risk of stenotic lesion formation and/or thrombotic clot generation.

In select embodiments of the present invention employing grafts or other synthetic structures, a combination of physical structures may be combined with the active use of one or more energies to enhance and manage patency. For example, in certain instances, the synthetic structure may be functionalized to promote the adhesion and proliferation of one or more desired cell types, e.g. endothelial cells. That is certain peptides, e.g. peptides containing arginine-glycine-aspartate amino acid moieties, have been shown to be beneficial in promoting endothelialization of vascular graft materials. Other materials and functionalizations may likewise be utilized to manage cell attachment or proliferation. For example, use of surface microfeatures may be employed to improve the flow of blood past the synthetic surface and thereby lessen the likelihood of undesired cell attachment and growth.

In the scope of the present invention, management of cellular attachment and proliferation may be further advanced through the use of one or more energies intended to manage one or more desired cellular processes, e.g. motility in a desired direction, and/or proliferation. In combination, the use of structures combining surface modification with delivered energies of the present invention may result in desired vascular performance that is better than either approach in isolation.

In yet other embodiments, one or more agents or materials may be introduced into the body as a whole and then by passage through the vasculature or by other means arrive at the target vascular region. Once at the intended vascular region, one or more energies, e.g. photonic energies, may be applied resulting in the activation of the agent or material. Such activation may include, but is not limited to, the release of a drug, the photo-induced transition of a drug from an inactive species to an active drug species, the creation of an active species then capable of forming therapeutic species, e.g. a photosensitizer, or the absorption of the applied energy and transformation into a second energy able to produce a therapeutic action, e.g. irradiation of a nanoparticle resulting localized thermal energy delivery.

In short, a variety of types, combinations and forms of additional energies, configurations and/or materials utilized with the energies of present invention are conceivable and accordingly, the present invention is not restricted to those described herein.

Sensors In select embodiments of the present invention, one or more sensors able to measure one or more parameters associated with vascular structure or related tissues may be employed to provide useful data supporting the present invention. Such sensor data may be useful in guiding the timing, levels and/or location of one or more of the energies intended to manage vascular patency within the scope of the present invention.

Examples of such sensors include but are not limited to, sensors able to detect change in vascular structure, including vessel wall dimensions, resiliency, fluid flow rates and/or blood component composition. For example, such sensors able to detect a thickening of the vessel wall without simultaneously detecting an overall increase in the outside circumference of the vessel may be useful for inferring the development of an occlusive growth, e.g. stenotic lesion, at this point in the vessel. Accordingly, a targeted delivery of one or more energies may then be delivered to this identified location in order to relieve the occlusive formation and restore vessel patency.

Examples of sensors include, but are not limited to, sensors employing electrical impedance signals, photonic signals, acoustic signals, pressure or pressure change signals, radio-wave signals, or chemical detection of specific agents or biological materials, e.g. hemoglobin. In addition, sensors may also utilize added agents, materials, nanostructures, compounds, etc. to better enable assessment of vascular patency. Such agents may include added dyes such that specific optical wavelengths may be employed to detect the presence and quantities of such materials.

In various forms of those embodiments of the present invention employing sensing means, the sensing means utilizes electrical currents between two or more electrodes located substantially in the vicinity of the target vascular region. Such electrical signals may include the use of impedance measurements in order to ascertain change in vessel luminal diameter and/or vessel wall thickness. For example, such sensors may enable the automated surveillance of thrombotic indicators and stenosis development in bypass grafts. By way of explanation, impedance signals are in general sensitive to the overall conductivity of the path followed by the electric currents. Changes in this pathway, such as will occur during stenosis lesion growth and progression, will thereby result in a change in to the electrical signature of the vessel in this region.

In addition, impedance signals may also enable further characterization of blood passage in vascular regions through comparative analysis of multiple regions of measurement. That is, the impedance measurement of blood has long been known to be influenced by the pulsatile movement or orientation of blood cells within the blood and therefore the magnitude and shape of the impedance measurements during pulsatile blood flow enables estimation of relative tumbling and velocity of blood cells. As a further refinement of this approach, use may be made of hematocrit determinations to adjust or enable comparisons of impedance measurements taken over a period of time.

In yet a further embodiment, use may be made of impedance measurements employing four electrodes axially arranged along a vessel and in substantial contact with the outside aspect of a vessel. For example, using all four electrodes, two electrodes may be employed to deliver an electrical signal, e.g. the outermost electrodes, and the other two electrodes employed to determine a voltage drop associated said signal in a vessel region defined by the two sensing electrodes. Such measurements enable determination of changes in luminal dimensions since the electrical signal may be considered to principally transit through the less resistive blood after passage through the higher resistive vessel walls. In contrast, measurement of impedance using the two outermost electrodes as both signal and sensing electrodes may be utilized to estimate the overall resistive path of the electrical signal, including the vessel walls. As the magnitude of this signal may reflect the higher resistance of the vessel wall, changes in wall thickness may be then determined through a set of measurements taken over a period of time, e.g. days or weeks. The preceding argument assumes that the spacing of the electrodes is such that the length of the electrical path in the blood affords substantially lower resistance than the passage of the signal through the vessel walls.

Alternative sensors may be employed within the scope of the present invention. For example, to pressure of blood flow may be determined using micro-machined cantilevers, or other pressure sensitive devices. Changes in said pressure determinations may then be employed to identify and track stenotic lesion formation and growth in a target vessel, as the blood flow velocity and downstream pressures may reflect the presence of a restriction in the lumen. One embodiment of this form of sensors may involve the inclusion of micron scale pressure devices in a useful pattern integrated into a film-like structure. Films with multiple pressure sensors may potentially be used to measure vascular pressure differentials, which are often critical in prediction of adverse events (e.g., artherosclerosis, thrombosis, etc.). These films may be positioned in a variety of vascular locations such as on the inner lumen of a graft, where pressure detection may be of higher sensitivity than on the outer aspect. The films may also be integrated with stents, stent grafts, or other intraluminal devices.

In preferred embodiments of forms of the present invention employing one or more sensing means, such sensors and associated controlling circuitry, e.g. power supply, amplifiers, A/D converters, microcontrollers, are substantially co-located in the structures or components of the device of the present invention employed for the delivery of one or more patency management energies. In various forms, some or all of the patency management energy sources, e.g. electrodes or photonic sources, may be employed for use in performing sensing functions. Likewise, circuitry, and power utilized for performing patency management activities may be used in whole or in part to perform sensing functions.

In further embodiments, sensor measurement data and/or analysis may be externally relayed to one or more devices substantially located on the outside of the body to enable external review and decision making Additional forms, types and combinations of sensors are readily conceivable for use within the scope of the present invention and accordingly, the scope of the present invention is not limited to those examples presented herein.

Comparators To effectively utilize sensor information as well as to adapt energy delivery to regions of vasculature as intended, various embodiments of the present invention may include one or more comparator functionalities. Such comparators may be contained as software within the circuitry of the device or located elsewhere and employ communication with the implanted device of the present invention as well as communicate, e.g. through displays and keyboard input, with attending individuals. In general terms, the analytical function of the comparator may be the determination of a change in vascular patency, e.g. positive or negative change, and/or the determination of the appropriate energy delivery response, e.g. energies to be applied, the timing of this application and/or the energy sources located in the vascular region to activate. In addition, a comparator may also enable of one or more additional therapeutic actions, e.g. activation of drug treatment, etc. Comparator activity, e.g. energy delivery and/or other actions may be taken automatically or may require approval/initiation by an individual such as a clinician.

In general terms, comparator algorithms enabling these determinations or other determinations within the scope of the present invention may also utilize additional data such as user age, gender, height, and vascular region or disease state in order to accomplish the desired determination. In certain other embodiments, other information, e.g. user or clinician inputted data such as co-morbidities, blood test results, physiological sensor results, stress test results, etc., or additional anthropometric or population-based data may also be included in one or more algorithms to improve accuracy and/or reliability. In still other forms of the invention, baseline parameters are established from one or more measurements such that deviation or trends from this baseline(s) may be determined and employed in subsequent calculations and estimations of patency and/or therapeutic response. This latter may be especially useful in those applications where patency may be at risk from surgical or medical procedures, e.g. immediately following surgery or after an extended period of time (weeks or months) following arteriovenous graft installation.

Sensor measurements utilized by comparator may include those made by the device of the present invention. In other embodiments, other sensors and sensor data are utilized by the comparator. Such sensors may include other sensors located within the body or sensors substantially located outside of the body. In still other forms of the invention, a combination of device sensor data as well as non-device sensor data are employed by the comparator in the determination of vascular patency and appropriate therapies to be applied.

In various embodiments, a plurality of sensor measurements may be taken to establish over short periods of time, e.g. minutes, such that these data may be averaged by the comparator to reduce uncertainties associated with measurement accuracies, e.g. signal noise attributable to motion artifacts. That is, one or more measurements may be taken, e.g. once every minute, and utilized for establishing values associated with this general point in time. In turn, several of these points taken over longer periods of time, e.g. hours, may then be utilized to provide determination of patency change in one or more vascular regions.

In alternative embodiments of the invention, less frequent or periodic measurements may be employed, e.g. measurements made through the use of handheld sensor platforms such as blood pressure assessment. These measurements may employ one or more measurement technologies and/or one or more body sites for supplying desired vascular patency data. In addition, additional sensors, e.g. heart rate, respiration, temperature, etc., or sensor measurements themselves may be employed to adjust the obtained measurements to minimize signal noise, position or motion artifacts.

In still other forms of the invention, measurements of several parameters reflecting an underlying physiological trait possibly associated with vascular patency, e.g. serum glucose levels, may be combined to form a more complete indication of the overall patency status. These assessments may be useful in the adjustment one or more parameters within the comparator, e.g. base patency or patency change associated with disease progression, to further tailor the described algorithm to the individual. These measured parameters may reflect both short term and long term status of the underlying physiological trait examined.

In addition, said information may be compiled over time to determine trends and enable estimation of time required for attaining one or more patency objectives, e.g. period of overall therapy application to accomplish desired healing post surgery, to generate population trends and data. In still other embodiments, the comparator may also automatically or upon demand review data sets to determine trends or patterns of patency, sensor measurement data, and/or therapy applications.

Comparator analysis may also include display and/or alerts to local devices and/or remote data management systems. Triggering events for such displays or alerts may be incorporated in the comparator memory as part of look up tables or based upon analysis of user data, e.g. rate of change of one or more parameters, loss of therapeutic energy functionality, etc. In addition, such presentations may also include one or more suggested courses of action or activity including preemptive actions to forestall loss of device functionality and/or patency.

In yet further embodiments, comparator activities may include reminders to the user to administer one or more medications, e.g. vasodilators or anticoagulants, or trigger the automatic delivery of one or more therapeutic agents. Conversely, delivery of one or more agents may be incorporated into therapy delivery for the adjustment of therapy, e.g. the timing and amount of delivered energy, based upon the metabolic status of the individual.

In form, the comparator may be substantially located within the implanted device of the present invention, e.g. with the controlling circuitry including processor, memory and power functionalities. In other embodiments, the comparator may be substantially located outside the body of the individual, e.g. in a local data collection unit and/or remote data management system. In yet other embodiments, comparator functionality is distributed between two or more of these elements, e.g. between the implanted device and the local data collection unit.

Comparator activities may be relatively simple, e.g. preprogrammed pulsatile current delivery for a period of time, or complex, able to adapt to changing vascular status indicated by sensor feedback and/or additional instructions or feedback from one or more external sources, e.g. clinician input. In short, multiple forms and methods of comparators and comparator activities are conceivable and the scope of the present invention is not limited to the examples presented herein.

### Examples of Use

In general terms, use of the device of this invention employs installation of the device into the body of a subject about or near a vascular structure for a period of time. Such periods of time may be comparatively short, e.g. hours or days, or comparatively long, e.g. months or years. Device installation may be performed in coordination with a medical procedure related to a vascular condition. In the context of this invention, the medical procedure may represent implantation of a medical structure, such as a stent, placement of vascular graft or creation of an arteriovenous fistula. In other embodiments, the device of the present invention may be installed at a time other than that of a medical procedure, e.g. to permit post-surgical recovery following the medical procedure.

Once installed, the device of the invention may be activated either upon command, e.g. manually activation of a switch such as one initiated by the contact of the device of the present invention with body fluids, or by command, e.g. remote wireless instruction. In certain embodiments of the invention, the activation and control of the device may be done in conjunction with or under automatic control of one or more body sensors, e.g. sensors detecting stenotic build-up.

Upon activation, in preferred embodiments of the invention, an electrical current may be passed substantially through vascular tissue and/or fluid components from one or more first electrodes to one or more second electrodes. The nature of this electrical current, including the amplitude, periodicity, frequency, duty cycle, and polarity may be based upon the instructions supplied by the control circuitry or as part of the construction of the apparatus itself, e.g. the polarity being set by battery contact orientation. Further control of the apparatus, including the cessation of activity, may be accomplished in a variety of fashions, including but not limited to, manual command, pre-set programming or received instructions.

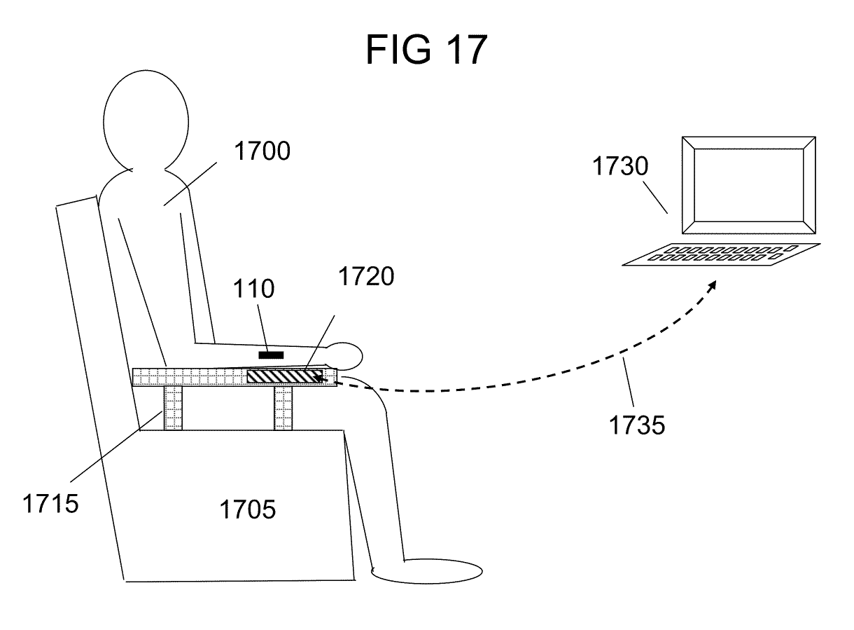

The device of the invention may be comprised as a single, self contained unit, having electrodes, control circuitry and power. Alternatively, the device may be a portion of a larger therapeutic system. An example of one form of this embodiment is presented in FIG. 17 showing individual 1700 sitting in chair 1705 with implanted device of the invention 110 located in the arm of the individual. Device control unit 1720 is located within the arm rest 1715 and is in wireless communication with implanted device 110. Said communications may include transmission of operational instructions to device 110 and the receipt of data regarding patency status/device. Also shown is wireless communication 1735 between data control unit 1720 and remote data management system 1730. Such embodiments may be useful employed in individuals suffering from chronic kidney failure who require hemodialysis. During these hemodialysis sessions this system employing wireless communication with the implanted device facilitates data acquisition for clinician oversight and review.

In alternative forms of this embodiment of the invention, the data collection unit may be configured as a unit placed periodically on or near the subject, e.g. as a handheld reader, or as a bedside unit, for the purpose of communication with implanted device. In still other embodiments of the invention, the data control unit also serves to supply at least a portion of power needed for operation to implanted device, e.g. through inductive coupling or radio-wave energy transmission.

In other embodiments of the invention, the device may form an element within a combined system for sensing one or more bioparameters and upon instruction and/or automated command, initiating a therapeutic response such as activation of one or more sets of energy delivery, e.g. electrodes, according to the method of the present invention. Such therapeutic response may also incorporate the application of additional therapies, e.g. other photonic energies, delivery of biological agents, etc. In particular, advantageous use may be made of one or more electrodes of the present invention to serve as sensors for detection of vascular performance, e.g. stenosis formation or fistula healing/maturation. As the forms and applications of the present invention may be readily appreciated to have a wide degree of variations, the nature and scope of the invention is accordingly not constrained to the examples presented herein.

Illustrative examples of potential uses of the present invention include:

Blood Vessel Patency Management In a preferred application of the present invention, the device of present invention utilizing the controlled delivery of electric currents may be employed in the management of neointimal hyperplasia associated with the narrowing or clogging of arteries. Such conditions may arise from a variety of medical procedures such as arising from the formation of an arteriovenous fistula and the effective management of the patency of these vessels enables improved health on the part of the patient and lower interventional costs and risks associated with surgical correction of the stenotic lesion.

In such applications, the device of the present invention may be installed during a surgical procedure revealing the target vessel region, e.g. the creation of the fistula. In greater detail, a structure containing one or more energy delivery sites is installed in substantial contact to the outside aspect of the vessel region in an effectively perivascular fashion and the circuit module/circuitry activated, e.g. by a magnetic switch or other control means prior to closing the placement site. The site is then closed with the device of the present invention functioning according to preprogrammed instructions in order support patency in the desired vascular region.

Graft Patency Extension of useful patency of vascular grafts, e.g. PTFE grafts employed for vascular access enabling dialysis in kidney failure patients or grafts employed in the treatment of peripheral artery disease, is another possible application of one or more devices of the invention. In one such application of the invention, the method and devices of the invention may be employed to accelerate healing of the anastomoses formed by the junction of the graft and the blood vessels, e.g. arteries or veins. In such embodiments, the nature of the electrical currents may be used to accelerate the ingrowth or migration of the vascular endothelial cells onto graft surfaces, thereby minimizing the ingrowth of fibroblasts or other non-desired cell types. The ability to promote an endothelial monolayer on a luminal aspect through the use of applied electric currents in vivo is novel and unique to this invention.

Alternatively, the applied currents may be used to minimize invasion of non-desired cell types, thereby allowing vascular endothelial cells having slower migration/growth characteristics the necessary time to grow into desired vascular regions.

In alternate applications, electric fields and currents may be used to manage neointimal growth directly within the graft or in vessel regions immediately adjacent to the graft, e.g. at one or more anastomosis.

In a related example, electric fields and currents may be employed to accelerate healing/maturation following venous surgery, e.g. endothelialization of grafts utilized in the treatment of peripheral artery disease, for the maturation of arteriovenous fistulas or the healing of vascular graft implants. In such applications, one or more sets of first and second electrodes may be positioned to advantageously accelerate movement of desired cell types to regions benefiting from said cells, e.g. vascular endothelial cells into wound regions. These electrodes may be subsequently be advantageously employed to minimize movement of undesired cell types in to these vascular regions and utilized by themselves or with additional first or second electrodes in order to accomplish the present invention.

Intraluminal Devices—Stents Implantable vascular devices possibly may benefit by employing the present invention to retard restenosis. Examples of such applications include the use with stents to minimize the likelihood of restenosis at the site of stent placement. One skilled in the art will readily recognize that additional devices and systems are conceivable and that the scope of this invention is not limited to those embodiments shown below.

Applications of the present invention is not be limited to these examples. Additional applications utilizing the controlled delivery of energy, e.g. electrical currents, to prevent, retard, or reverse the formation of a stenotic lesion or to advantageously accelerate healing of a target vascular are conceivable. Accordingly, the scope of applications utilizing the present invention is not limited to the examples presented herein.

What is claimed is:

1. The management of vascular patency in a vascular tissue region utilizing an implanted device having:
at least one first electrically conductive element configured to be positioned in the vicinity of a vascular region and having a first surface for the delivery of an electric current to vascular tissue;
at least one second electrically conductive element also having a first surface and that is configured to be placed elsewhere; and,
at least one electrical current generating source electrically connected to said first and second conductive elements;
wherein the activation of said electrical current generating source results in the passage of an effectively directional electric current between the first surface of a first conductive element and the first surface of a second conductive element and is intended for the management of vascular patency in the vascular region.

2. The device of claim 1 wherein the current generating source is activated over an extended period of time in order to accomplish the management of vascular patency.

3. The device of claim 2 wherein the electric current is delivered in a pulsatile fashion.

4. The device of claim 1 wherein the first and second conductive elements are configured as electrodes.

5. The device of claim 4 wherein the electrodes are positioned on a structure located substantially about an outer aspect of a blood vessel.

6. The device of claim 4 wherein the first surfaces of the electrodes are configured to be substantially in contact with at least a portion of the outer aspect of a vascular tissue region.

7. The device of claim 4 wherein said first and second electrodes have effectively different first surface areas.

8. The device of claim 4 wherein a plurality of first and second electrodes are adjustably activated.

9. The device of claim 4 wherein at least one first electrode may be reversibly utilized as a second electrode.

10. The device of claim 4 wherein said first electrode is substantially located in synthetic vessel.

11. The device of claim 4 wherein said first electrode is substantially located in a vascular tissue lumen.

12. The device of claim 1 also incorporating one or more sensor functionalities useful for the determination of vascular patency.

13. The device of claim 12 wherein the sensor functionality utilizes electrical impedance.

14. The management of vascular patency in a mammalian body utilizing an implantable device having:
at least one energy delivery source configured to be positioned in the vicinity of a region of a vascular tissue structure;
at least one energy delivery control structure electrically connected to said energy delivery source; and
wherein the activation of said energy delivery control structure results in the release of at least one energy from said energy delivery source for the purpose of managing vascular patency in the vascular region.

15. The device of claim 13 wherein said energy is one of electrical, acoustic, photonic, thermal or radio-wave energies.

16. The device of claim 13 also incorporating one or more sensor functionalities useful for the determination of vascular patency.

17. A method for managing vascular patency by:
the placement of at least one first electrode in the vicinity of a vascular structure region;
the placement of at least one second electrode elsewhere; and
at least one electrical power and control module electrically connected to at least one first and at least one second electrode;
wherein the passage of an electrical current between at least one first electrode and at least one second electrode, utilizing electrical currents supplied by the electric power and control module, results in a desired management of vascular patency in the vascular structure region.

18. The method of claim 17 wherein said electrical currents are delivered in a pulsatile fashion.

19. The method of claim 18 wherein said pulsatile currents are delivered over an extended period of time.

20. The method of claim 17 wherein said first electrodes may be substantially located in a vascular tissue lumen, an artificial vascular structure or are located in substantial contact with the outer aspect of a blood vessel.
